(19) United States
(12) Patent Application Publication
(10) Pub. No.: US 2022/0313040 A1
(43) Pub. Date: Oct. 6, 2022

(54) CLEANING DEVICE INCLUDING VACUUM CLEANER AND DUST COLLECTING STATION

(71) Applicant: SAMSUNG ELECTRONICS CO., LTD., Suwon-si (KR)

(72) Inventors: Ingyu CHOI, Suwon-si (KR); Seehyun KIM, Suwon-si (KR); Kihwan KWON, Suwon-si (KR); Hyeoncheol KIM, Suwon-si (KR); Hyunju LEE, Suwon-si (KR); Hyunho LEE, Suwon-si (KR); Seungryong CHA, Suwon-si (KR)

(73) Assignee: SAMSUNG ELECTRONICS CO., LTD., Suwon-si (KR)

(21) Appl. No.: 17/709,533

(22) Filed: Mar. 31, 2022

Related U.S. Application Data
(63) Continuation of application No. PCT/KR2022/003715, filed on Mar. 17, 2022.

(30) Foreign Application Priority Data
Mar. 30, 2021 (KR) 10-2021-0041451
Dec. 23, 2021 (KR) 10-2021-0186459
Feb. 8, 2022 (KR) 10-2022-0016437

(51) Int. Cl. A47L 9/14; A47L 5/28; A47L 9/16; A47L 9/00; A47L 9/02; B08B 1/00; B08B 1/04; B08B 5/02; A46B 13/02

(52) U.S. Cl. CPC A47L 9/149; A47L 5/28; A47L 9/1683; A47L 9/0063; A47L 9/02; B08B 1/002; B08B 1/04; B08B 5/02; A46B 13/02; A46B 2200/3033

(57) ABSTRACT

A cleaning device including a vacuum cleaner having an intake portion suctioning foreign substances and a dust collecting container collecting the foreign substances. The cleaning device also including a dust collecting station connected to the dust collecting container. The dust collecting station having a station body, a seating portion including an opening, a suction device configured to generate a suction airflow so that the foreign substances in the dust collecting container are discharged through the opening, and a collecting portion arranged between the seating portion and the suction device and configured to collect the foreign substances that are caused to flow through the opening of the seating portion from the inside of the dust collecting container by the suction airflow.

### CROSS-REFERENCE TO RELATED APPLICATION

This application is a continuation application, under 35 U.S.C. § 111(a), of International Patent Application No. PCT/KR2022/003715, filed on Mar. 17, 2022, which claims priority under 35 U.S.C. § 119 to Korean Patent Applications No. 10-2021-0041451, filed on Mar. 30, 2021, 10-2021-0186459, filed on Dec. 23, 2021, and 10-2022-001637, filed on Feb. 8, 2022 in the Korean Intellectual Property Office, the disclosures of which are incorporated herein by reference.

### BACKGROUND

### 1. Field

The disclosure relates to a cleaning device including a vacuum cleaner and a dust collecting station, and more specifically, to a cleaning device including a vacuum cleaner and a dust collecting station capable of automatically cleaning an intake portion of the vacuum cleaner.

### 2. Description of the Related Art

A vacuum cleaner is an apparatus equipped with a fan motor for generating a suction force to suction foreign substances, such as dust, together with air by the suction force generated by the fan motor through an intake portion. In addition, the vacuum cleaner is a device designed to separate foreign substances contained in the suctioned air from the air and collect the foreign substances such that cleaning is performed.

In the intake portion of the vacuum cleaner, foreign substances, such as various types of dust, are accumulated or hair is tangled. In order to clean the foreign substances, the user may disassemble the intake portion and manually remove the foreign substances. Alternatively, the foreign substances may be removed using an airflow that flows into the intake portion through a separate exhaust device.

However, when the user manually removes foreign substances from the intake portion, cleaning may not be performed frequently and the usability may be lowered. In addition, in the method of introducing an airflow into the intake portion through a separate exhaust device, removal of dust and foreign substances may not be completely performed.

### SUMMARY

According to an aspect of the disclosure, there is provided a cleaning device including: a vacuum cleaner including an intake portion to suction foreign substances into the vacuum cleaner, and a dust collecting container to collect the foreign substances through centrifugal rotation separation, and a dust collecting station connectable to the dust collecting container and configured to remove the foreign substances collected in the dust collecting container, the dust collecting station including: a station body; a seating portion including an opening to communicate with an inside of the dust collecting container while the dust collecting station is connected to the dust collecting container, a suction device configured to generate a suction airflow so that the foreign substances in the dust collecting container are suctioned through the opening of the seating portion; a collecting portion arranged between the seating portion and the suction device and configured to collect the foreign substances that are suctioned through the opening of the seating portion; a base provided at a lower side of the station body to support the station body and configured to allow the suction airflow to flow therein; and an exhaust portion connected to the base and formed to allow the suction airflow therein, wherein the exhaust portion includes an exhaust hole through which the suction airflow is exhausted to the intake portion so that the foreign substances remaining on the intake portion are separated from the intake portion.

The a circulation flow path extends along the exhaust portion, the station body, the base, and the vacuum cleaner.

The exhaust portion may include: an accommodation frame on which the intake portion is seated; a motor portion mounted on the accommodation frame; and a cleaning unit having one end coupled to the motor portion to separate dust accumulated on the intake portion, the cleaning unit arranged inside the accommodation frame to face a bottom surface of the intake portion.

The cleaning unit may include: a rotation shaft configured to rotate by receiving power from the motor portion; and a brush extending in a radial direction with respect to the rotation shaft and provided to rotate, the brush provided to separate dust on the bottom surface of the intake portion.

The cleaning unit may include a cleaner including the rotation shaft and the brush, and the cleaner may be provided in plural to sweep off the dust in the intake portion.

The cleaning unit may include: a drive shaft configured to rotate by receiving power from the motor portion; a support shaft arranged in parallel with the driving shaft and fixed to the accommodation frame; and a moving plate allowing the driving shaft and the support shaft to pass therethrough and provided to be in contact with the intake portion, the moving plate provided to be slidably movable with respect to the accommodation frame according to rotation of the driving shaft.

The moving plate may include a cutting member mounted on one surface of the moving plate that faces the intake portion.

The base may include: a communication frame including a communication flow path forming a part of the circulation flow path; and a support frame connected to the communication frame to be in contact with an installation surface of the dust collecting station.

The dust collecting station may further include a filter portion provided at a lower side of the collecting portion to filter air that has passed through the collecting portion, and the air that has passed through the filter portion may be introduced into the communication flow path of the base.

The exhaust portion may include an accommodation frame in which the intake portion is accommodated, the accommodation frame being integrally formed with the communication frame for air to circulate.

The accommodation frame may include a front seating portion that is bent to allow a portion of a front side of the intake portion to be seated thereon.

The exhaust portion may be provided to be detachable with respect to the base.

The exhaust portion may be provided to move between a first position in which the exhaust hole is inserted into the base and a second position in which the exhaust hole protrudes outside the base such that the suction airflow is exhausted to the intake portion.

The dust collecting station may include: a first flow path formed inside the station body and connected from the seating portion to the suction device; and a second flow path connected from the suction device to the base, wherein one end of the second flow path may be formed in the exhaust hole.

The exhaust portion may further include a cover forming an upper surface of the exhaust portion, and the cover may form at least a portion of the base when the exhaust portion is arranged in the first position.

According to an aspect of the disclosure, there is provided a cleaning device including: a vacuum cleaner including an intake portion suctioning foreign substances, a dust collecting container collecting the foreign substances through centrifugal rotation separation, and an extension pipe connecting the dust collecting container to the intake portion; and a dust collecting station connected to the dust collecting container to remove the foreign substances collected in the dust collecting container, wherein the dust collecting station includes: a station body; a seating portion including an opening provided to communicate with an inside of the dust collecting container, the seating portion configured to seat the dust collecting container thereon; a suction device configured to form a suction airflow such that the foreign substances in the dust collecting container are discharged through the opening of the seating portion; a collecting portion arranged between the seating portion and the suction device and configured to collect the foreign substances that are caused to flow through the opening of the seating portion from the inside of the dust collecting container by the suction airflow; a base provided at a lower side of the station body to support the station body; and an exhaust portion including an exhaust hole formed to allow the suction airflow to be exhausted to the intake portion such that the foreign substances remaining on the intake portion are separated from the intake portion, wherein the exhaust portion is provided to move between a first position in which the exhaust hole is inserted into the base and a second position in which the exhaust hole protrudes outside the base such that the suction airflow is exhausted to the intake portion.

The dust collecting station may include: a first flow path formed inside the station body and connected from the seating portion to the suction device; and a second flow path connected from the suction device to the base, wherein one end of the second flow path may be formed in the exhaust hole.

The exhaust portion may further include a cover forming an upper surface of the exhaust portion, and the cover may form at least a portion of the base when the exhaust portion is arranged in the first position.

The exhaust portion may include a guide that forms the exhaust hole and guides the suction airflow such that the suction airflow flows into the exhaust hole.

The guide may be arranged inside the base when the exhaust portion is arranged in the first position, and may be provided to protrude outside of the base when the exhaust portion is arranged in the second position.

The dust collecting station may be provided such that, in response to the exhaust portion being arranged in the second position and the suction device being driven, foreign substances collected in the dust collecting container are caused to flow into the collecting portion by the suction airflow and then collected into the dust collecting container, and the suction flow passed through the collecting portion is introduced into the intake portion so as to pass through the extension pipe together with the foreign substances remaining in the intake portion and then flows back to the collecting portion.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a perspective view illustrating a state in which a vacuum cleaner is coupled to a dust collecting station of a cleaning device according to one embodiment of the disclosure;

FIG. 2 is a side cross-sectional view of FIG. 1;

FIG. 3 is a perspective view illustrating a base and an exhaust portion of the cleaning device according to the one embodiment of the disclosure;

FIG. 4 is an exploded perspective view of FIG. 3;

FIG. 5 is a cross-sectional view illustrating an operation of the exhaust portion of the cleaning device according to the one embodiment of the disclosure;

FIG. 6 is a cross-sectional view illustrating an operation of the exhaust portion of the cleaning device according to the one embodiment of the disclosure;

FIG. 7 is a front perspective view illustrating an exhaust portion of a cleaning device according to another embodiment of the disclosure;

FIG. 8 is a bottom perspective view of FIG. 7;

FIG. 9 is an exploded perspective view of FIG. 7;

FIG. 10 is a cross-sectional view illustrating an operation of the exhaust portion of the cleaning device according to the another embodiment of the disclosure;

FIG. 11 is a cross-sectional view illustrating an operation of the exhaust portion of the cleaning device according to the another embodiment of the disclosure;

FIG. 12 is a perspective view illustrating a state in which a vacuum cleaner is coupled to a dust collecting station of a cleaning device according to still another embodiment of the disclosure;

FIG. 13 is an enlarged cross-sectional view illustrating a portion of an exhaust portion of FIG. 12;

FIG. 14 is an exploded perspective view illustrating the exhaust portion of the cleaning device according to the still another embodiment of the disclosure;

FIG. 15 is a front perspective view illustrating an exhaust portion of a cleaning device according to still another embodiment of the disclosure;

FIG. 16 is a bottom perspective view of FIG. 15;

FIG. 17 is a view illustrating an operation of the exhaust portion, which is seen from the front with a bottom frame shown in FIG. 15 removed;

FIG. 18 is a view illustrating an operation of an exhaust portion, which is seen from the front, with a bottom frame shown in FIG. 15;

FIG. 19 is a perspective view illustrating a state in which a vacuum cleaner is coupled to a dust collecting station of a cleaning device according to still another embodiment of the disclosure;

FIG. 20 is a side cross-sectional view of FIG. 19;

FIG. 21 is a view illustrating a state in which the exhaust portion of the dust collecting station shown in FIG. 19 is inserted into a base;

FIG. 22 is a view illustrating a state in which the exhaust portion of the dust collecting station shown in FIG. 19 is withdrawn from a base;

FIG. 23 is a side cross-sectional view of FIG. 22;

FIG. 24 is a view showing the exhaust portion and the base of the dust collecting station of FIG. 19;

FIG. 25 is a cross-sectional view illustrating an operation of a cleaning unit of the dust collecting station of FIG. 19;

FIG. 26 is a cross-sectional view illustrating an operation of a cleaning unit of the dust collecting station of FIG. 19;

FIG. 27 is a schematic block diagram illustrating a control flow of the dust collecting station shown in FIG. 19; and

FIG. 28 is a cross-sectional view illustrating a base and an exhaust portion of the cleaning device according to the still another embodiment of the disclosure.

### DETAILED DESCRIPTION

The embodiments set forth herein and illustrated in the configuration of the disclosure are only the most preferred embodiments and are not representative of the full technical spirit of the disclosure, so it should be understood that they may be replaced with various equivalents and modifications at the time of the disclosure.

Throughout the drawings, like reference numerals refer to like parts or components.

The terminology used herein is for the purpose of describing particular embodiments only and is not intended to limit the disclosure. It is to be understood that the singular forms “a,” “an,” and “the” include plural references unless the context clearly dictates otherwise. It will be further understood that the terms “include”, “comprise” and/or “have” when used in this specification, specify the presence of stated features, integers, steps, operations, elements, and/or components, but do not preclude the presence or addition of one or more other features, integers, steps, operations, elements, components, and/or groups thereof.

The terms including ordinal numbers like “first” and “second” may be used to explain various components, but the components are not limited by the terms. The terms are only for the purpose of distinguishing a component from another. Thus, a first element, component, region, layer or section discussed below could be termed a second element, component, region, layer or section without departing from the teachings of the disclosure. Descriptions shall be understood as to include any and all combinations of one or more of the associated listed items when the items are described by using the conjunctive term “˜ and/or ˜,” or the like.

Therefore, it is an object of the disclosure to provide a cleaning device including an exhaust portion capable of simultaneously cleaning an intake portion and emptying a dust collecting container as a vacuum cleaner is docked on a dust collecting station.

It is another object of the disclosure to provide a cleaning device capable of physically separating foreign substances by a friction between an exhaust portion and an intake portion and immediately collecting the separated foreign substances into a collecting portion of a dust collecting station.

It is another object of the disclosure to provide a cleaning device capable of achieving slimness of a dust collecting station and improving aesthetics by providing an exhaust portion to be accommodated in a base of the dust collecting station.

The technical objectives of the disclosure are not limited to the above, and other objectives may become apparent to those of ordinary skill in the art based on the following descriptions.

Hereinafter, embodiments according to the disclosure will be described in detail with reference to the accompanying drawings.

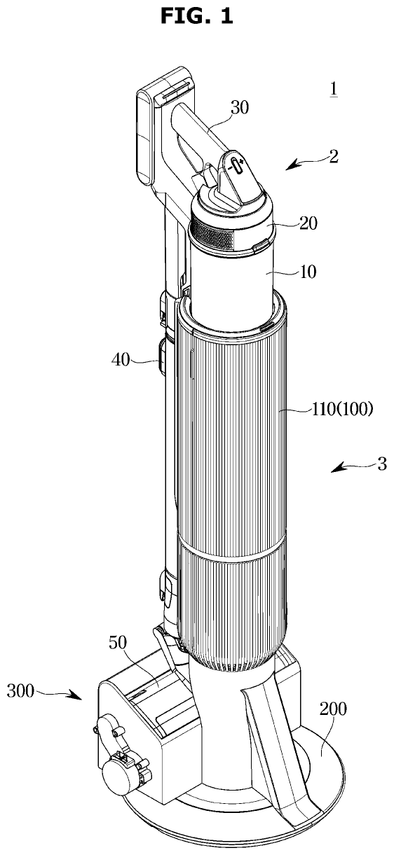

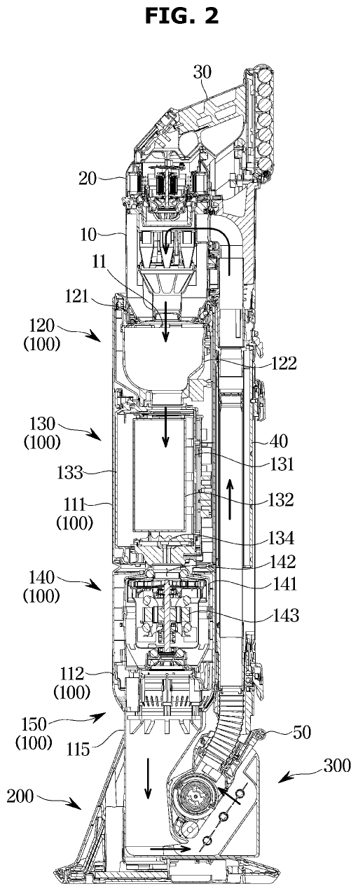

FIG. 1 is a perspective view illustrating a state in which a vacuum cleaner of a cleaning device is coupled to a dust collecting station according to the one embodiment of the disclosure. FIG. 2 is a side cross-sectional view of FIG. 1.

Referring to FIGS. 1 and 2, a cleaning device 1 may include a vacuum cleaner 2 and a dust collecting station 3 provided to seat the vacuum cleaner 2 thereon.

The vacuum cleaner 2 may include a cleaner main body 20 and a dust collecting container 10 detachably coupled to the cleaner main body 20. The dust collecting container 10 may be provided to collect foreign substances moved into the vacuum cleaner 2. A dust collecting container door 11 may be rotatably coupled to the dust collecting container 10 of the vacuum cleaner 2. The dust collecting container door 11 closes the dust collecting container 10 when the user normally uses the vacuum cleaner 2 to prevent dust inside the dust collecting container 10 from escaping to the outside. Thereafter, when the user mounts the vacuum cleaner 2 on the dust collecting station 3 and the dust collecting station 3 is driven, the dust collecting container door 11 may open the dust collecting container 10. The method of opening and closing the dust collecting container door 11 may be implemented in various ways.

The vacuum cleaner 2 may include an intake portion 50 and an extension pipe 40.

The intake portion 50 may be provided to suction external foreign substances into the vacuum cleaner 2. The extension pipe 40 may be provided to connect the cleaner main body 20 and the intake portion 50 to form a passage through which foreign substances move.

The suctioned foreign substances may be moved to the dust collecting container 10 through the cleaner main body 20, and collected into the dust collecting container 10. That is, the extension pipe 40 may be provided to connect the intake portion 50 and the dust collecting container 10.

The vacuum cleaner 2 according to the embodiment of the disclosure has been described and illustrated as being provided such that the cleaner main body 20 is mounted on the front of the extension pipe 40. In addition, the dust collecting container 10 connected to the cleaner main body 20 may also be arranged on the front of the extension pipe 40. In this case, the front may refer to an area in front of the user when the user holds the vacuum cleaner 2. However, the disclosure is not limited thereto, and the cleaner main body 20, the extension pipe 40, and the dust collecting container 10 may be arranged in various positions.

The vacuum cleaner 2 may include a handle 30 connected to the vacuum cleaner main body 20 and the extension pipe 40. Specifically, the handle 30 may be coupled to the upper side of the cleaner main body 20 and the extension pipe 40. The user may hold the handle 30 to move the vacuum cleaner 2 and perform cleaning on a cleaning surface. The arrangement of the handle 30 may also be provided to be arranged at various positions.

With the vacuum cleaner 2 seated on the dust collecting station 3, the vacuum cleaner 2 may be docked onto the dust collecting station 3 such that the longitudinal direction of the extension pipe 40 is arranged to approximately be in a direction corresponding to the upper side and lower side direction. Accordingly, an exhaust portion 300 of the dust collecting station 3 to be described below may be arranged adjacent to the intake portion 50.

Referring to FIG. 2, the dust collecting station 3 may include a main body 100 and a base 200 supporting the main body 100.

The station body 100 may include a housing 110 provided to cover each component arranged inside the station body 100 to be described below. The housing 110 may form the external appearance of the station body 100. The housing 110 may be provided to have a long axis extending in one direction. Specifically, the housing 110 may be preferably provided in a shape extending in the upper side and lower side direction. In addition, the surface of the housing 110 may be formed to have a curved surface. As an example, the dust collecting station 3 may be provided in a substantially cylindrical shape.

The housing 110 may include a first housing 111 and a second housing 112.

The first housing 111 may form the external appearance of an upper portion of the station body 100. The second housing 112 may form an external appearance of a lower portion of the station body 100.

The first housing 111 and the second housing 112 may be provided to have a long axis extending in one direction. Specifically, the first housing 111 and the second housing 112 may preferably be provided in a shape extending in the upper side and lower side direction. In addition, the surfaces of the first housing 111 and the second housing 112 may be formed to have a curved surface.

However, the configuration of the first housing 111 and the second housing 112 is not limited thereto, and the first housing 111 and the second housing 112 may be integrally formed with each other.

The dust collecting station 3 may include a depression (not shown). The depression (not shown) may be formed by a portion of the dust collecting station 3 depressed from the outside to the inside of the first housing 111 and the second housing 112. The depression (not shown) may accommodate a portion of the extension pipe 40 of the vacuum cleaner 2. With such a configuration, the vacuum cleaner 2, when mounted on the dust collecting station 3, may be stored in a more compact size.

The housing 110 may include a connecting portion 115 arranged at a lower side of the housing 110 and connected to the base 200. The connecting portion 115 may be provided by at least a portion of the housing 110 depressed in a direction toward the central axis of the housing 110 such that the intake portion 50 of the vacuum cleaner 2 is accommodated in the central axis direction of the housing 110 in a state in which the vacuum cleaner 2 is mounted on the dust collecting station 3. With such a configuration, the vacuum cleaner 2, when mounted on the dust collecting station 3, may be stored in a more compact size. However, the disclosure is not limited thereto, and the connecting portion 115 may be provided to extend in the upper side and lower side direction to have the same vertical shape as the housing 110.

The dust collecting station 3 may include a charging portion (not shown) that is electrically connected to a battery (not shown) of the vacuum cleaner 2 such that the battery (not shown) of the vacuum cleaner 2 is charged when the vacuum cleaner 2 is docked onto the dust collecting station 3.

The dust collecting station 3 may include a seating portion 120 arranged inside the housing 110 and on which the dust collecting container 10 is seated. The seating portion 120 may include a seating portion opening 121 provided to communicate with the inside of the dust collecting container 10. The seating portion opening 121 may be provided in a substantially hollow annular shape. The seating portion 120 may be accommodated in the first housing 111. The seating portion 120 may include a duct 122.

The duct 122 of the seating portion 120 may be provided to allow foreign substances collected in the dust collecting container 10 of the vacuum cleaner 2 to move to a dust bag 132 which will be described below.

The duct 122 may be provided at an upper side thereof with an open portion that is connected to the seating portion opening 121. With such a configuration, when the vacuum cleaner 2 is mounted on the dust collecting station 3, foreign substances from the dust collecting container 10 may be moved into the duct 122. That is, the open portion of the duct 122 may be provided corresponding to the diameter of the dust collecting container door 11 that opens and closes the dust collecting container 10. However, the disclosure is not limited thereto, and the open portion of the duct 122 may be formed larger than the diameter of the dust collecting container door 11.

The duct 122 may be provided at a lower side thereof with a dust outlet. Accordingly, the foreign substances discharged from the dust collecting container 10 of the vacuum cleaner 2 may move into the duct 122, and through the dust outlet finally move to a collecting portion 130 which will be described below.

The dust collecting station 3 may include the collecting portion 130 arranged inside the housing 110.

The collecting portion 130 may include a mounting frame 131, a dust bag 132 detachably coupled to the inside of the mounting frame 131, and a dust bag cover 133 coupled to the mounting frame 131 at the front of the dust bag 132.

The mounting frame 131 may include a dust inlet formed at an upper portion thereof. The dust inlet allows the mounting frame 131 to communicate with the dust outlet formed in the duct 122 of the seating portion 120. Accordingly, foreign substances moved through the seating portion 120 may be collected in the collecting portion 130.

To the inside of the mounting frame 131, the dust bag 132 may be coupled. Through the dust inlet of the mounting frame 131, foreign substances passed through the seating portion 120 may be collected into the dust bag 132.

The dust bag cover 133 may be provided to cover the front of the dust bag 132. The dust bag cover 133 may be coupled to the mounting frame 131 to accommodate the dust bag 132 therein.

The collecting portion 130 may include a filtering portion 134.

The filtering portion 134 may be arranged at a lower side of the mounting frame 131 and the dust bag cover 133. Foreign substances that failed to enter the dust bag 132 may be accumulated on the filtering portion 134. Accordingly, the filtering portion 134 may be provided to prevent foreign substances from entering a suction device 140 positioned below the collecting portion 130.

The dust collecting station 3 may include the suction device 140 and a filter portion 150 arranged inside the housing 110.

The suction device 140 may include a motor cover 141. The suction device 140 may form a suction airflow provided to allow the foreign substances collected in the dust collecting container 10 to flow into the dust collecting station 3.

The motor cover 141 may include an airflow hole 142. The suction device 140 may include a driving device 143. Accordingly, the suction force generated from the driving device 143 of the suction device 140 may reach the collecting portion 130 and the seating portion 120.

The driving device 143 may generate a suction force by providing power. The driving device 143 may include components, such as a motor and a fan. That is, the driving device 143 may cause a suction airflow to be generated in the dust collecting station 3.

The filter portion 150 may include a filter therein. When the driving device 143 of the suction device 140 forms an airflow to suction air from the upper side of the dust collecting station 3, the air passed through the suction device 140 may pass through the filter portion 150. The filter portion 150 may be provided at a lower side of the collecting portion 130 to filter the air passed through the collecting portion 130. The air passed through the filter portion 150 may flow into the base 200, which will be described below.

The base 200 may be provided at a lower side of the main body 100 to support the main body 100.

The base 200 may include the exhaust portion 300 in which the intake portion 50 of the vacuum cleaner 2 is accommodated. The exhaust portion 300 may be provided to exhaust air to remove foreign substances remaining in the intake portion 50. The exhaust portion 300 may be integrally formed with the base 200.

Details of the exhaust portion 300 will be described below.

As shown by the arrow in FIG. 2, the main body 100 and the base 200 of the dust collecting station 3 may form a circulation flow path, through which air is circulated, together with the vacuum cleaner 2. Details thereof will be described below.

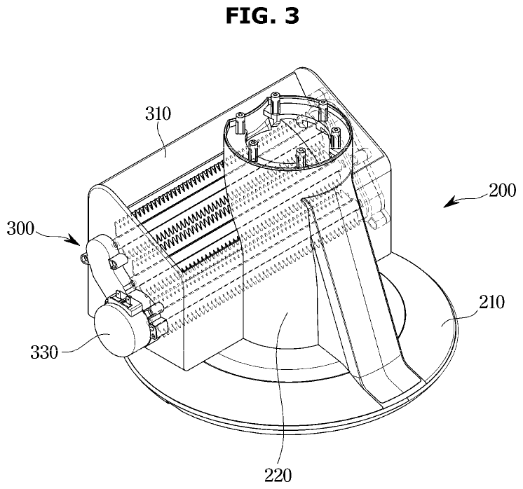

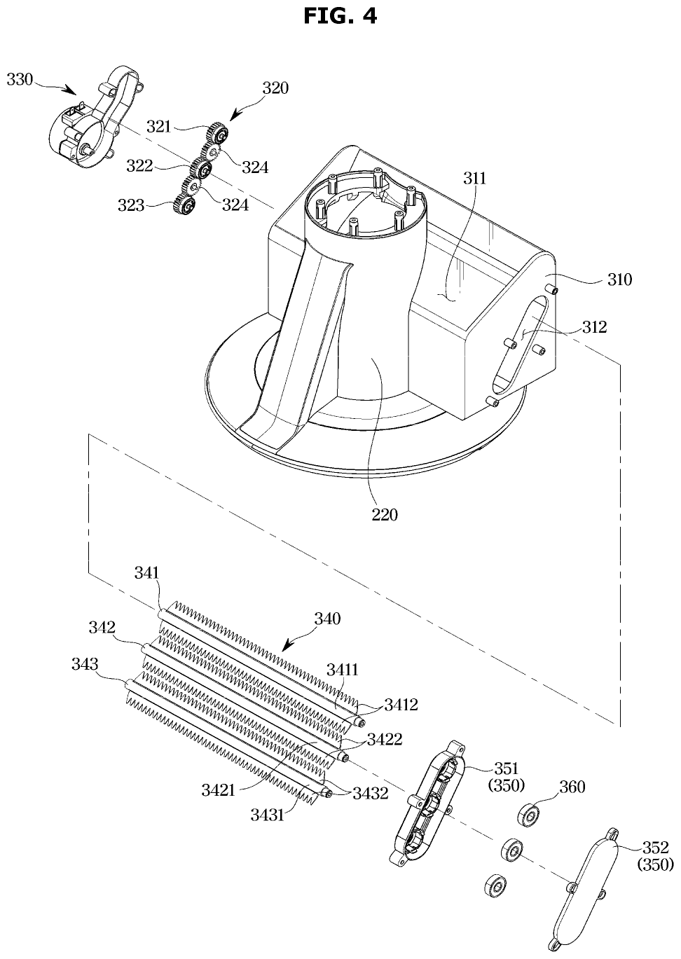

FIG. 3 is a perspective view illustrating a base and an exhaust portion of the cleaning device according to the one embodiment of the disclosure. FIG. 4 is an exploded perspective view of FIG. 3;

Referring to FIGS. 3 and 4, the base 200 may include a communication frame 220 and a support frame 210.

The communication frame 220 may be connected to the second housing 112. The communication frame 220 may be coupled to the second housing 112. The communication frame 220 may be mounted on the lower portion of the main body 100.

The support frame 210 may be connected to the communication frame 220 to be in contact with an installation surface of the dust collecting station 3. The support frame 210 may be provided such that a portion in contact with the installation surface is fixed, and an upper side of the contact portion is rotatable. Alternatively, the support frame 210 may be provided in a fixed form as one body.

The dust collecting station 3 may include the exhaust portion 300. The exhaust portion 300 may be provided to accommodate the intake portion 50 of the vacuum cleaner 2 to separate foreign substances remaining in the intake portion 50. The exhaust portion 300 may be formed in the base 200 to form a circulation flow path, through which air flows, together with the main body 100.

The exhaust portion 300 may include an accommodation frame 310.

The accommodation frame 310 may accommodate and seat the intake portion 50 therein.

The accommodation frame 310 may be provided in a shape that is open approximately at an upper side thereof. The accommodation frame 310 may include an accommodation space 311 formed inside to accommodate the intake portion 50.

The accommodation frame 310 may be formed at a side surface with an opening 312. The opening 312 allows a cleaning unit 340, which will be described below, to be rotatably mounted with respect to the accommodation frame 310.

The accommodation frame 310 may include a front seating portion 313. The front seating portion 313 may be provided to seat a portion of the front side of the intake portion 50 thereon. That is, the front seating portion 313 may be provided in a shape corresponding to the shape of a nozzle housing 51 of the intake portion 50.

The accommodation frame 310 may be integrally formed with the base 200. More specifically, the exhaust portion 300 may be integrally formed with the communication frame 220 and the support frame 210.

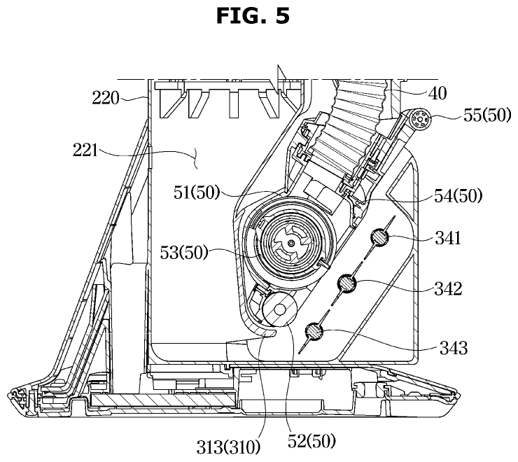

With such a configuration, the accommodation frame 310 may be provided to form a circulation flow path together with the communication frame 220. More specifically, a communication flow path (221 in FIG. 5) of the communication frame 220 may communicate with the accommodation space 311 of the accommodation frame 310. The front seating portion 313 of the accommodation frame 310 may divide the communication flow path 221 of the communication frame 220 from the accommodation space 311 of the accommodation frame 310.

The exhaust portion 300 may include a motor portion 330.

The motor portion 330 may be mounted on the accommodation frame 310.

More specifically, the motor portion 330 may be mounted on one side of the accommodation frame 310. The motor portion 330 may include a driving motor (not shown). Power may be generated through the driving motor (not shown).

The exhaust portion 300 may include a gear unit 320.

The gear unit 320 may be mounted on the motor portion 330. The gear unit 320 may include a first gear 321, a second gear 322, a third gear 323, and a transmission gear 324. More specifically, the transmission gear 324 may be provided between the first gear 321 and the second gear 322 and may be provided between the second gear 322 and the third gear 323. Accordingly, the transmission gear 324 may be provided in plural.

The exhaust portion 300 may include the cleaning unit 340.

The cleaning unit 340 may have one end coupled to the motor portion 330 to separate dust accumulated in the intake portion 50. The cleaning unit 340 may be arranged inside the accommodation frame 310 to face the bottom surface of the intake portion 50.

The cleaning unit 340 may include a first cleaner 341, a second cleaner 342, and a third cleaner 343.

The first cleaner 341 may include a first rotation shaft 3411 and a first brush 3412. The first rotation shaft 3411 may be rotated by receiving power from the motor portion 330. More specifically, the first rotation shaft 3411 may be rotated by receiving power from the motor portion 330 through the first gear 321.

The first brush 3412 may extend radially from the first rotation shaft 3411. The first brush 3412 may be provided as a pair to improve the cleaning ability of the exhaust portion 300. However, the number of the first brushes 3412 is not limited thereto. The first brush 3412 may be rotated according to the rotation of the first rotation shaft 3411 to separate dust on the bottom surface of the intake portion 50.

The second cleaner 342 may include a second rotation shaft 3421 and a second brush 3422. The second rotation shaft 3421 may be rotated by receiving power through the second gear 322. More specifically, the rotation of the first gear 321 may be transmitted to the second gear 322 through the transmission gear 324. Accordingly, as the second gear 322 rotates, the second rotation shaft 3421 may also be rotated.

The second brush 3422 may extend radially from the second rotation shaft 3421. The second brush 3422 may be provided as a pair. However, the number of the second brushes 3422 is not limited thereto. The second brush 3422 may be rotated according to the rotation of the second rotation shaft 3421 to separate dust from the bottom surface of the intake portion 50.

The third cleaner 343 may include a third rotation shaft 3431 and a third brush 3432. The third rotation shaft 3431 may be rotated by receiving power through the third gear 323. In more detail, the rotation of the first gear 321 may be transmitted to the second gear 322 through the transmission gear 324, and the rotation of the second gear 322 may be transmitted to the third gear 323 through the transmission gear 324. Accordingly, as the third gear 323 rotates, the third rotation shaft 3431 may be also be rotated.

The third brush 3432 may extend radially from the third rotation shaft 3431. The third brush 3432 may be provided as a pair. However, the number of the third brushes 3432 is not limited thereto. The third brush 3432 may be rotated according to the rotation of the third rotation shaft 3431 to separate dust from the bottom surface of the intake portion 50.

In FIGS. 3 and 4, the cleaning unit 340 is illustrated and described as including the first cleaner 341, the second cleaner 342, and the third cleaner 343, but the number of cleaners is not limited thereto and may be changed. Accordingly, the number of gears of the gear unit 320 may also be changed.

The exhaust portion 300 may include a coupling frame 350 and a bearing 360.

The coupling frame 350 may be mounted on the accommodation frame 310. More specifically, the coupling frame 350 may be mounted on the other side of the accommodation frame 310 that is the opposite side to the motor portion 330. Through the coupling frame 350, the other end of the cleaning unit 340 may be rotatably supported by passing through the opening 312 of the accommodation frame 310.

The bearing 360 may be arranged inside the coupling frame 350 to assist the cleaning unit 340 to rotate smoothly. The number of bearings 360 may be provided to correspond to the number of the cleaners of the cleaning unit 340.

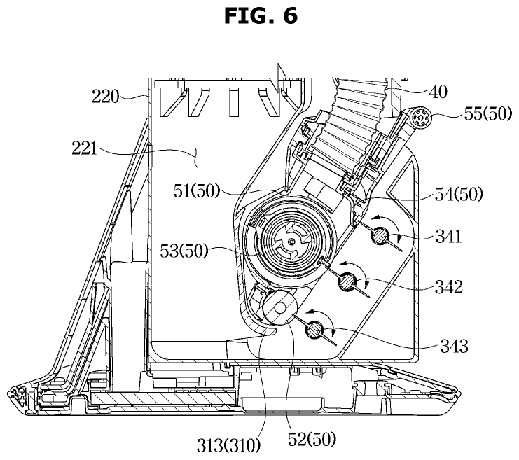

FIGS. 5 and 6 are cross-sectional views illustrating an operation of a nozzle cleaner of the cleaning device according to the one embodiment of the disclosure.

Hereinafter, an operation of the exhaust portion 300 of the cleaning device will be described with reference to FIGS. 5 and 6.

The communication frame 220 may include the communication flow path 221 to form a portion of the circulation flow path formed by the vacuum cleaner 2 and the dust collecting station 3. The front seating portion 313 of the accommodation frame 310 may be bent to seat a portion of the front side of the intake portion 50 thereon. The accommodation frame 310 may be formed integrally with the communication frame 220 for air to be circulated.

The intake portion 50 may include a nozzle housing 51.

The nozzle housing 51 may be formed to have a bottom surface that is open. The nozzle housing 51 of the vacuum cleaner 2 may be provided to be connected to the extension pipe 40. More specifically, the nozzle housing 51 may be provided to be connected to a flex tube of the extension pipe 40. The nozzle housing 51 has an inner portion with a space in which various parts are mounted.

The intake portion 50 may include a roller 52. The roller 52 may be provided on the front side of the nozzle housing 51. The roller 52 may be rotatably provided to allow dust on the cleaning surface in contact with the intake portion 50 of the vacuum cleaner 2 to be moved to an inner side of the nozzle housing 51.

The intake portion 50 may include a cleaning roller 53.

The cleaning roller 53 may be provided to collect dust by rotating at a rear side of the roller 52. The cleaning roller 53 may be formed of cotton flannel material such that fine dust on the cleaning surface is attached to the cleaning roller 53. However, the material of the cleaning roller 53 is not limited thereto.

The intake portion 50 may include a pusher 54.

The pusher 54 may be provided to push away dust collected on the rear side of the cleaning roller 53 according to a user's manipulation. When the user applies a force to move the vacuum cleaner 2 forward, dust is aggregated between the cleaning roller 53 and the pusher 54 by the rotation of the cleaning roller 53, and the pusher 54 scrapes off the aggregated dust such that the dust may be moved to the dust collecting container 10 through the extension pipe 40 by the suction force of the vacuum cleaner 2.

The exhaust portion 300 may be arranged at a lower side of the nozzle housing 51 to sweep off dust and foreign substances from the roller 52, the cleaning roller 53, and the pusher 54 of the intake portion 50.

More specifically, the first cleaner 341 sweeps off dust and foreign substances from the pusher 54, the second cleaner 342 sweeps off dust and foreign substances from the cleaning roller 53, and the third cleaner 343 sweeps off the dust and foreign substances from the roller 52. That is, the first cleaner 341, the second cleaner 342, and the third cleaner 343 may rotate to remove dust and foreign substances from the bottom surface of the intake portion 50.

At the same time as the exhaust portion 300 is physically removing dust from the intake portion 50, the dust collecting station 3 may be operated. Hereinafter, a process of forming a circulation flow path according to the operation of the dust collecting station 3 will be described.

When the dust collecting station 3 is operated, the suction device 140 of the dust collecting station 3 generates a suction force. In this case, the dust collecting container door 11 of the dust collecting container 10 may open the dust collecting container 10.

However, the disclosure is not limited thereto, and the dust collecting container door 11 may be opened by a separate configuration, and when the dust collecting container 10 is seated on the seating portion 120, the dust collecting container door 11 pressed by the seating portion 120 may be preliminary opened.

The dust previously collected in the dust collecting container 10 may pass through the seating portion 120. The dust passed through the seating portion 120 may move to the collecting portion 130 and accumulate in the collecting portion 130.

The air passed through the collecting portion 130 may pass through the filter portion 150 and flow into the communication flow path 221 of the communication frame 220. The air flowing into the communication flow path 221 may flow into the accommodation frame 310 of the exhaust portion 300.

At this time, the exhaust portion 300 may operate for the cleaning unit 340 to separate dust and foreign substances from the intake portion 50. Accordingly, the air moved into the accommodation space 311 of the accommodation frame 310 flows back into the intake portion 50, and dust and foreign substances separated from the intake portion 50 are caused to flow along the extension pipe 40 of the vacuum cleaner 2.

The dust and foreign substances moved along the extension pipe 40 may pass through the dust collecting container 10 of the vacuum cleaner 2 and then finally gather into the collecting portion 130 of the dust collecting station 3. With such a configuration, a circulation flow path between the vacuum cleaner 2 and the dust collecting station 3 may be formed.

Accordingly, the air in the circulation flow path of the cleaning device according to the embodiment of the disclosure moving from the dust collecting container 10 of the vacuum cleaner 2 and passing through the main body 100 and the base 200 of the dust collecting station 3 may flow through the exhaust portion 300, and then back into the intake portion 50 of the vacuum cleaner 2. With such a configuration, only when the user mounts the vacuum cleaner 2 on the dust collecting station 3, the intake portion 50 may be cleaned without a separate action.

In addition, the cleaning of the intake portion 50 is performed at the same time as the dust collecting container 10 of the vacuum cleaner 2 is emptied, so that the vacuum cleaner 2 is always kept clean without dust or foreign substances remaining in the intake portion 50.

In addition, because the intake portion 50 is cleaned with the clean air that passed through the filter portion 150 of the dust collecting station 3, the cleaning performance of the intake portion 50 may be improved, and dust may not be newly attached to the intake portion 50.

In addition, because dust separated from the intake portion 50 is not collected in the dust collecting container 10 of the vacuum cleaner 2, but is directly collected by the collecting portion 130 of the dust collecting station 3, the dust collecting performance of the dust collecting container 10 may be maintained for a long time.

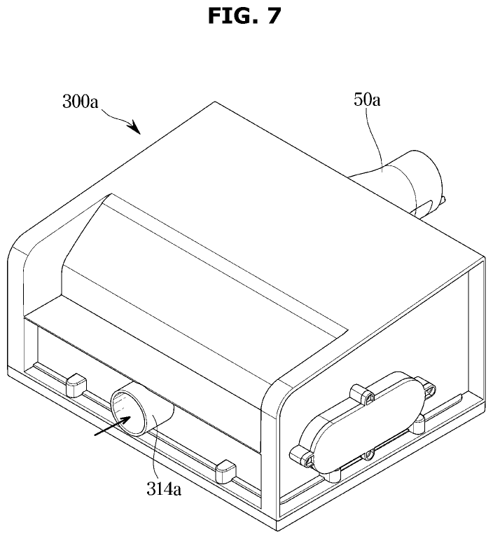

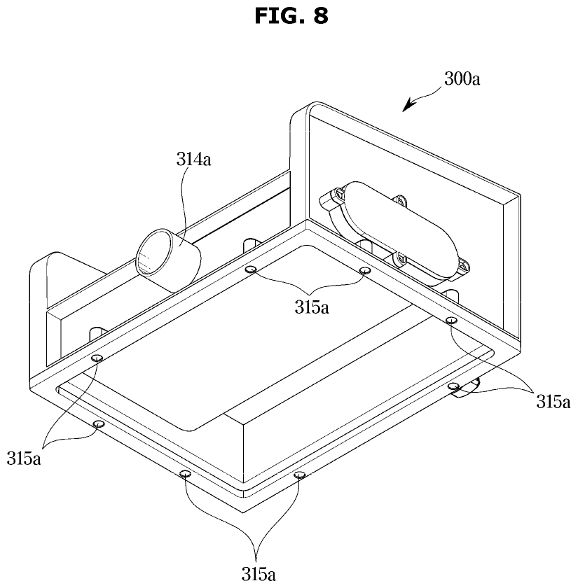

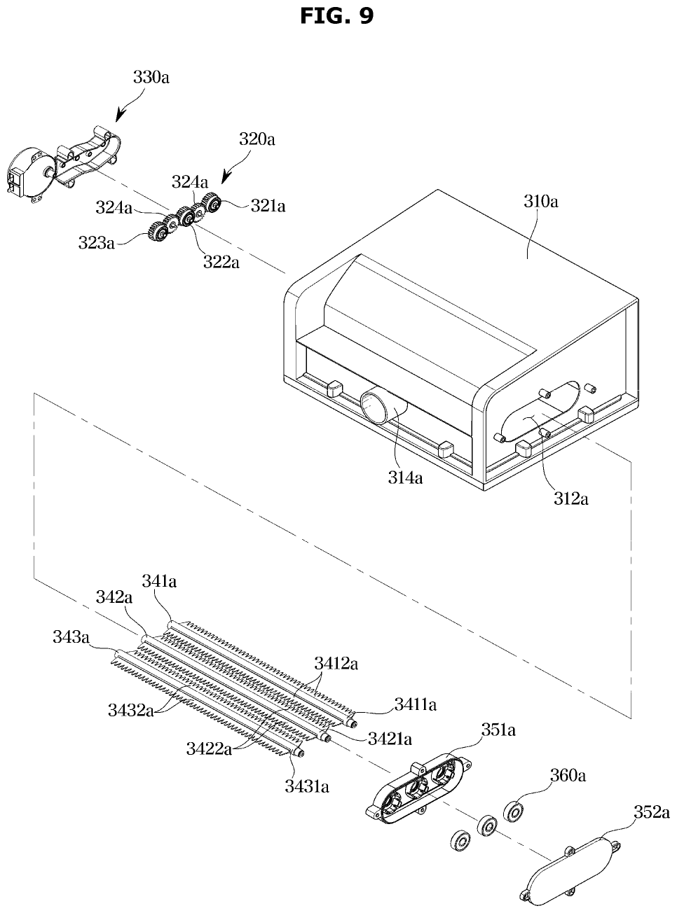

FIG. 7 is a front perspective view illustrating an exhaust portion of a cleaning device according to another embodiment of the disclosure. FIG. 8 is a bottom perspective view of FIG. 7. FIG. 9 is an exploded perspective view of FIG. 7.

The following description will be made on some features that are different from those of the exhaust portion 300 of the cleaning device according to the one embodiment of the disclosure. Components not described in the following may be used in the same manner as those of the cleaning device according to the one embodiment of the disclosure, and assigned the same reference numerals.

Referring FIGS. 7 to 9, an exhaust portion 300a of a cleaning device according to another embodiment of the disclosure may be detachably provided on a base 200 of a dust collecting station 3.

The exhaust portion 300a may include a communication portion 314a formed in a nozzle accommodation frame 310a to form a circulation flow path together with the dust collecting station 3. The communication portion 314a may be provided in a tubular shape with an interior that is open such that air flows therein. However, the shape of the communication portion 314a is not limited thereto.

In addition, the exhaust portion 300a may include a coupling portion 315a formed on the bottom surface thereof. The coupling portion 315a may be provided such that the exhaust portion 300a is coupled to the base 200 of the dust collecting station 3 using a separate fastening member.

The exhaust portion 300a may include the nozzle accommodation frame 310a.

The nozzle accommodation frame 310a may accommodate and seat an intake portion 50a therein.

The nozzle accommodation frame 310a may be provided in a shape that is open approximately at one side. The nozzle accommodation frame 310a may include a nozzle accommodation space formed inside to accommodate the intake portion 50a.

The nozzle accommodation frame 310a may have a side portion with an opening 312a. The opening 312a may allow a cleaning unit 340a, which will be described below, to be rotatably mounted with respect to the nozzle accommodation frame 310a.

The nozzle accommodation frame 310a may include a seating portion 313a. The seating portion 313a may be provided to seat a portion of the front side of the intake portion 50a thereon. That is, the seating portion 313a may be provided in a shape corresponding to the shape of a nozzle housing 51a of the intake portion 50a.

The nozzle accommodation frame 310a may be provided to form a circulation flow path together with the dust collecting station 3.

The exhaust portion 300a may include a motor portion 330a.

The motor portion 330a may be mounted on the nozzle accommodation frame 310a. More specifically, the motor portion 330a may be mounted on one side of the nozzle accommodation frame 310a. The motor portion 330a may include a driving motor. Power may be generated through the drive motor.

The exhaust portion 300a may include a gear unit 320a.

The gear unit 320a may be mounted on the motor portion 330a. The gear unit 320a may include a first gear 321a, a second gear 322a, a third gear 323a, and a transmission gear 324a. More specifically, the transmission gear 324a may be provided between the first gear 321a and the second gear 322a, and may be provided between the second gear 322a and the third gear 323a. Accordingly, the transmission gear 324a may be provided in plural.

The exhaust portion 300a may include a cleaning unit 340a.

The cleaning unit 340a may have one end coupled to the motor portion 330a to separate dust accumulated in the intake portion 50a. The cleaning unit 340a may be arranged inside the nozzle accommodation frame 310a to face the bottom surface of the intake portion 50a.

The cleaning unit 340a may include a first cleaner 341a, a second cleaner 342a, and a third cleaner 343a.

The first cleaner 341a may include a first rotation shaft 3411a and a first brush 3412a. The first rotation shaft 3411a may be rotated by receiving power from the motor portion 330a. More specifically, the first rotation shaft 3411a may be rotated by receiving power from the motor portion 330a through the first gear 321a.

The first brush 3412a may extend radially from the first rotation shaft 3411a. The first brush 3412a may be provided as a pair to improve the cleaning ability of the exhaust portion 300a. However, the number of the first brushes 3412a is not limited thereto. The first brush 3412a may be rotated according to the rotation of the first rotation shaft 3411a to separate dust from the bottom surface of the intake portion 50a.

The second cleaner 342a may include a second rotation shaft 3421a and a second brush 3422a. The second rotation shaft 3421a may be rotated by receiving power through the second gear 322a. More specifically, the rotation of the first gear 321a may be transmitted to the second gear 322a through the transmission gear 324a. Accordingly, as the second gear 322a rotates, the second rotation shaft 3421a may also be rotated.

The second brush 3422a may extend radially from the second rotation shaft 3421a. The second brushes 3422a may be provided as a pair. However, the number of the second brushes 3422a is not limited thereto. The second brush 3422a may be rotated according to the rotation of the second rotation shaft 3421a to separate dust from the bottom surface of the intake portion 50a.

The third cleaner 343a may include a third rotation shaft 3431a and a third brush 3432a. The third rotation shaft 3431a may be rotated by receiving power through the third gear 323a. More specifically, the rotation of the first gear 321a may be transmitted to the second gear 322a through the transmission gear 324a, and the rotation of the second gear 322a may be transmitted to the third gear 323a through the transmission gear 324a. Accordingly, as the third gear 323a rotates, the third rotation shaft 3431a may also be rotated.

The third brush 3432a may extend radially from the third rotation shaft 3431a. The third brush 3432a may be provided as a pair. However, the number of the third brushes 3432a is not limited thereto. The third brush 3432a may be rotated according to the rotation of the third rotation shaft 3431a to separate dust from the bottom surface of the intake portion 50a.

In FIG. 9, the cleaning unit 340a is illustrated and described as including the first cleaner 341a, the second cleaner 342a, and the third cleaner 343a, but the number of cleaners is not limited thereto and may be changed. Accordingly, the number of gears of the gear unit 320a may also be changed.

The exhaust portion 300a may include a coupling frame 350a and a bearing 360a.

The coupling frame 350a may be mounted on the nozzle accommodation frame 310a. More specifically, the coupling frame 350a may be mounted on the other side of the nozzle accommodation frame 310a that is the opposite side to the motor portion 330a. Through the coupling frame 350a, the other end of the cleaning unit 340a may be rotatably supported by passing through the opening 312a of the nozzle accommodation frame 310a.

The bearing 360a may be arranged inside the coupling frame 350a to assist the cleaning unit 340a to rotate smoothly. The number of bearings 360a may be provided to correspond to the number of the cleaners of the cleaning unit 340a.

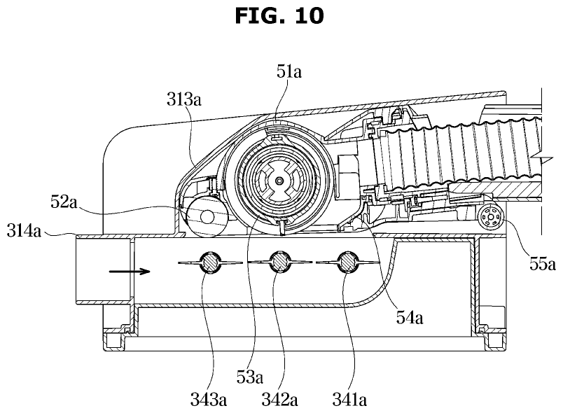

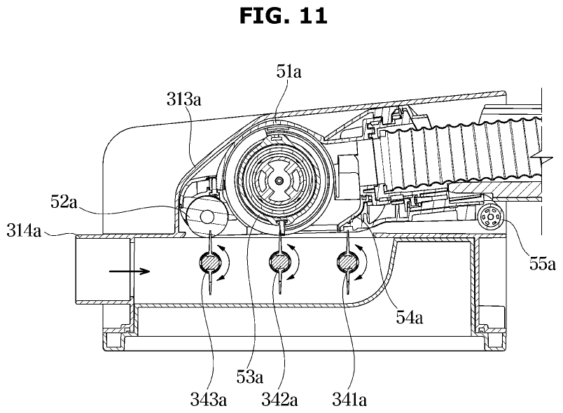

FIGS. 10 and 11 are cross-sectional views illustrating an operation of the exhaust portion 300a of the cleaning device according to the another embodiment of the disclosure.

Hereinafter, the operation of the exhaust portion 300a of the cleaning device according to the another embodiment of the disclosure will be described with reference to FIGS. 10 and 11.

The nozzle accommodation frame 310a may include the communication portion 314a to form a portion of a circulation flow path formed by the vacuum cleaner and the dust collecting station.

The seating portion 313a of the nozzle accommodation frame 310a may be bent to seat a portion of the front side of the intake portion 50a thereon. The nozzle accommodation frame 310a may be provided to communicate with the communication portion 314a.

The intake portion 50a may include the nozzle housing 51a.

The nozzle housing 51a may be formed to have a bottom surface that is openable. The nozzle housing 51a of the vacuum cleaner may be provided to be connected to the extension pipe. More specifically, the nozzle housing 51a may be provided to be connected to a flex tube of the extension pipe. The nozzle housing 51a may have an inner portion with a space in which various parts are mounted.

The intake portion 50a may include a rolling portion 52a. The rolling portion 52a may be provided on the front side of the nozzle housing 51a. The rolling portion 52a may be rotatably provided to allow dust on a cleaning surface in contact with the intake portion 50a of the vacuum cleaner to be moved to an inner side of the nozzle housing 51a.

The intake portion 50a may include an exhaust portion 53a.

The exhaust portion 53a may be provided to collect dust by rotating at the rear side of the rolling portion 52a. The exhaust portion 53a may be formed of a cotton flannel material such that fine dust on the cleaning surface is attached to the exhaust portion 53a. However, the material of the exhaust portion 53a is not limited thereto.

The intake portion 50a may include a push portion 54a.

The push portion 54a may be provided to push away the dust collected on the rear side of the exhaust portion 53a according to a user's manipulation. When the user applies a force to move the vacuum cleaner forward, dust is aggregated between the exhaust portion 53a and the push portion 54a by the rotation of the exhaust portion 53a, and the push portion 54a scrapes off the dust such that the dust may be moved to the dust collecting container through the extension pipe by the suction force of the vacuum cleaner.

The exhaust portion 300a may be arranged at a lower side of the nozzle housing 51a to sweep off dust and foreign substances from the rolling portion 52a, the exhaust portion 53a, and the push portion 54a of the intake portion 50a.

More specifically, the first cleaner 341a may sweep off dust and foreign substances from the push portion 54a, the second cleaner 342a may sweep off dust and foreign substances from the exhaust portion 53a, and the third cleaner 343a may sweep of dust and foreign substances of the rolling portion 52a. That is, the first cleaner 341a, the second cleaner 342a, and the third cleaner 343a may rotate to remove dust and foreign substances from the bottom surface of the intake portion 50a.

At the same time as the exhaust portion 300a is physically removing dust from the intake portion 50a, the dust collecting station 3 may be operated. Hereinafter, a process of forming a circulation flow path according to the operation of the dust collecting station 3 will be described.

When the dust collecting station 3 is operated, a driving portion of the dust collecting station generates a suction force. In this case, the cover of the dust collecting container may open the dust collecting container.

The dust previously collected in the dust collecting container may pass through a duct portion. The dust passed through the duct portion may move to the collecting portion and accumulate in the collecting portion.

The air passed through the collecting portion may pass through the filter portion and flow to the communication portion 314a of the nozzle accommodation frame 310a. The air moved to the communication portion 314a may flow into the nozzle accommodation frame 310a of the exhaust portion 300a.

At this time, the exhaust portion 300a may operate for the cleaning unit 340a to separate dust and foreign substances from the intake portion 50a. Accordingly, the air moved into the nozzle accommodation space of the nozzle accommodation frame 310a flows back into the intake portion 50a, and dust and foreign substances separated from the intake portion 50a are caused to flow along the extension pipe of the vacuum cleaner.

Therefore, the exhaust portion 300a of the cleaning device according to the another embodiment of the disclosure may be detachably coupled to the dust collecting station 3, unlike the exhaust portion 300 of the cleaning device according to the one embodiment of the disclosure. Accordingly, the exhaust portion 300a may include the separate communication portion 314a.

In addition, the aspect of the disclosure is not limited thereto, and the user may seat the intake portion 50a on the exhaust portion 300a separately provided on the dust collecting station 3 and drive the vacuum cleaner and the exhaust portion 300a. That is, the intake portion 50a may be cleaned the exhaust portion 300a and the separated dust and foreign substances may be suctioned using the motor of the vacuum cleaner.

In addition, the intake portion 50a may be cleaned by manually rotating the cleaning unit 340a by a user without supplying the exhaust portion 300a with power.

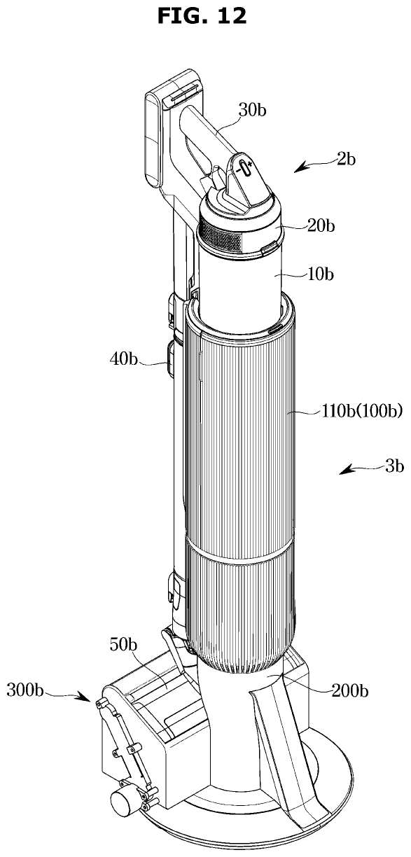

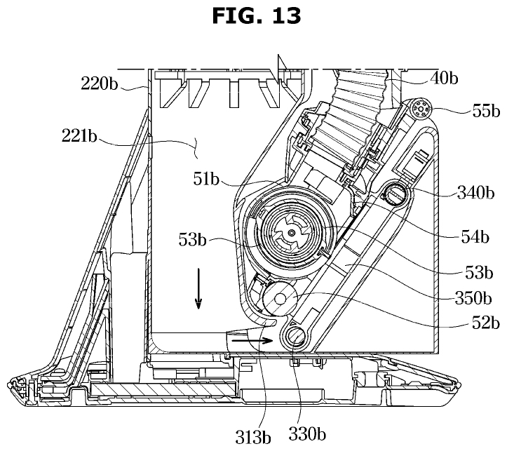

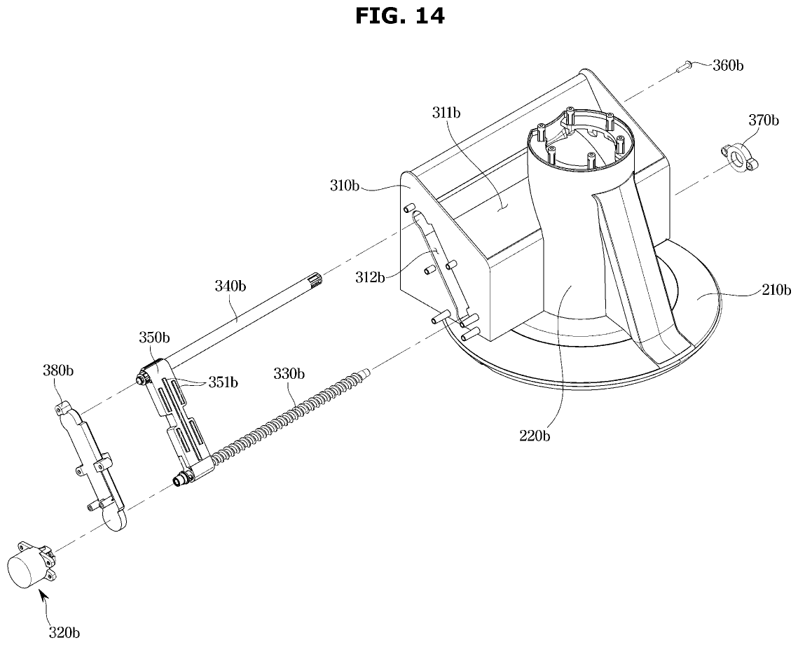

FIG. 12 is a perspective view illustrating a state in which a vacuum cleaner is coupled to a dust collecting station of a cleaning device according to still another embodiment of the disclosure. FIG. 13 is an enlarged cross-sectional view illustrating an exhaust portion of FIG. 12. FIG. 14 is an exploded perspective view illustrating the exhaust portion of the cleaning device according to the still another embodiment of the disclosure.

The following description will be made base on some features that are different from those of the cleaning device according to the above embodiment of the disclosure.

Referring to FIG. 12, a vacuum cleaner 2b may include a vacuum cleaner main body 20b and a dust collecting container 10b detachably coupled to the vacuum cleaner main body 20b. The dust collecting container 10b may be provided to collect foreign substances moved into the vacuum cleaner 2b.

The vacuum cleaner 2b may include an intake portion 50b and an extension pipe 40b.

The intake portion 50b may be provided to suction external foreign substances into the vacuum cleaner 2b. The extension pipe 40b may be provided to connect the vacuum cleaner main body 20b and the intake portion 50b to form a passage through which foreign substances move.

The suctioned foreign substances may be moved to the dust collecting container 10b through the vacuum cleaner main body 20b and collected in the dust collecting container 10b. That is, the extension pipe 40b may be provided to connect the intake portion 50b and the dust collecting container 10b.

The vacuum cleaner 2b may include a handle 30b connected to the vacuum cleaner main body 20b and the extension pipe 40b. Specifically, the handle 30b may be coupled to the upper portion of the vacuum cleaner main body 20b and the extension pipe 40b. The user may hold the handle 30b to move the vacuum cleaner 2b and perform cleaning on a cleaning surface.

When the vacuum cleaner 2b is mounted on a dust collecting station 3b, the intake portion 50b may be accommodated in an exhaust portion 300b formed in the dust collecting station 3b.

Hereinafter, the exhaust portion 300b of the cleaning device according to the still another embodiment of the disclosure will be described with reference to FIGS. 12 to 14.

The exhaust portion 300b may include an accommodation frame 310b.

The accommodation frame 310b may accommodate and seat the intake portion 50b therein.

The accommodation frame 310b may be provided in a shape that is open approximately on an upper side thereof. The accommodation frame 310b may include an accommodation space 311b formed therein to accommodate the intake portion 50b.

The accommodation frame 310b may have a side portion with an opening 312b. The opening 312b may allow a cleaning unit, which will be described below, to be slidably mounted with respect to the accommodation frame 310b.

The accommodation frame 310b may include a front seating portion 313b. The front seating portion 313b may be provided to seat a portion of the front side of the intake portion 50b thereon. That is, the front seating portion 313b may be provided in a shape corresponding to the shape of a nozzle housing 51b of the intake portion 50b.

The accommodation frame 310b may be integrally formed with a base 200. More specifically, the exhaust portion 300b may be integrally formed with a communication frame 220b and a support frame 210b.

With such a configuration, the nozzle accommodation frame 310b may be provided to form a circulation flow path together with the communication frame 220b. More specifically, a communication flow path 221b of the communication frame 220b may communicate with the accommodation space 311b of the accommodation frame 310b. The front seating portion 313b of the accommodation frame 310b may divide the communication flow paths 221b of the communication frame 220b from the accommodation space 311b of the accommodation frame 310b.

The exhaust portion 300b may include a motor portion 320b.

The motor portion 320b may be mounted on the accommodation frame 310b. More specifically, the motor portion 320b may be mounted on one side of the accommodation frame 310b. The motor portion 320b may include a driving motor. Power may be generated through the drive motor.

The exhaust portion 300b may include a cleaning unit. The cleaning unit according to still another embodiment of the disclosure may include a driving shaft 330b and a support shaft 340b.

The driving shaft 330b may be provided to rotate by receiving power from the motor portion 320b. The driving shaft 330b may have a screw thread formed on the outer surface thereof to guide the movement of a moving plate 350b, which will be described below. The driving shaft 330b may be arranged to extend lengthwise along the long side direction of the intake portion 50b.

The support shaft 340b may be arranged parallel to the driving shaft 330b. The support shaft 340b may be fixed to the accommodation frame 310b.

The cleaning unit may include the moving plate 350b.

The moving plate 350b may have one end that allows the driving shaft 330b to pass therethrough, and the other end that allows the support shaft 340b to pass therethrough.

The moving plate 350b may be provided to be movable along the extension direction of the driving shaft 330b and the support shaft 340b. More specifically, the moving plate 350b may be provided to be slidably movable with respect to the accommodation frame 310b according to the rotation of the driving shaft 330b. The one end of the moving plate 350b, through which the driving shaft 330b passes, has an inner surface with a groove portion (not shown) that has a shape corresponding to the screw thread of the driving shaft 330b. With such a configuration, as the driving shaft 330b rotates, the moving plate 350b may be linearly moved.

The moving plate 350b may include a cutting member 351b mounted on one surface thereof facing the intake portion 50b. In the intake portion 50b, foreign substances, such as hair, etc., tangled or aggregated inside the intake portion 50b may exist. Accordingly, as the foreign substances are cut using the cutting member 351b, the foreign substances may be more easily removed from the intake portion 50b.

The cutting member 351b may injure a user when the intake portion 50b is not mounted on the exhaust portion 300b. Accordingly, the cutting member 351b may be provided to protrude from the one surface of the moving plate 350b only when the intake portion 50b is mounted on the accommodation frame 310b through a separate elastic device (not shown).

Alternatively, the cutting member 351b may be controlled to protrude from the one surface of the moving plate 350b only when the intake portion 50b is mounted on the accommodation frame 310b by a separate sensing device.

In addition to the cutting member 351, a separate dust removal member having a frictional force to remove dust from the intake portion 50b may be formed on the one surface of the moving plate 350b. The type and material of the dust removal member may be variously provided as long as it can remove the dust of the intake portion 50b.

In addition, although not shown separately, various parts for cleaning the intake portion 50b may be mounted on the one surface of the moving plate 350b.

The cleaning unit may include a coupling frame 380b. The coupling frame 380b may be mounted on one side of the accommodation frame 310b for coupling to the driving shaft 330b and the supporting shaft 340b. The motor portion 320b may be coupled to the coupling frame 380b.

The cleaning unit may include a coupling member 360b and a support cap 370b.

The coupling member 360b may be mounted on the other side of the accommodation frame 310b to fix the support shaft 340b to the accommodation frame 310b. The support cap 370b may be mounted on the other side of the accommodation frame 310b to rotatably support the driving shaft 330b with respect to the accommodation frame 310b.

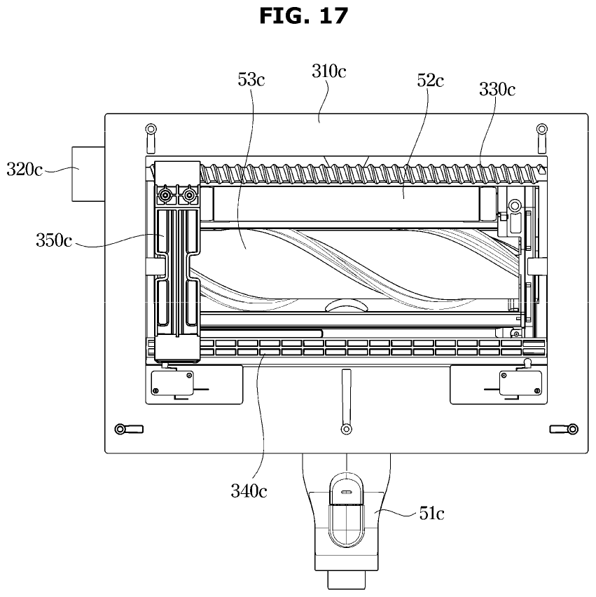

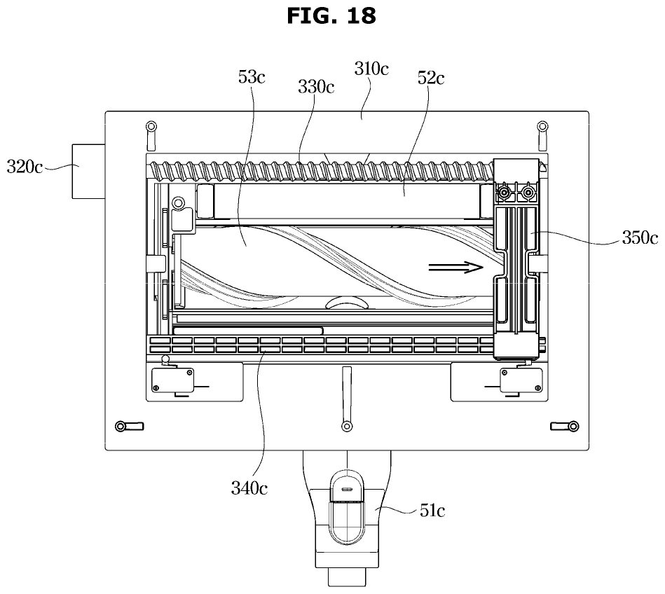

The operation of the exhaust portion 300b is the same as that of FIGS. 17 and 18, which will be described below, and thus details thereof will be described below.

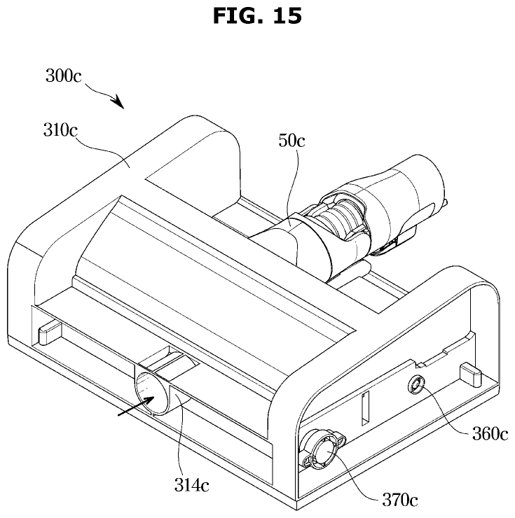

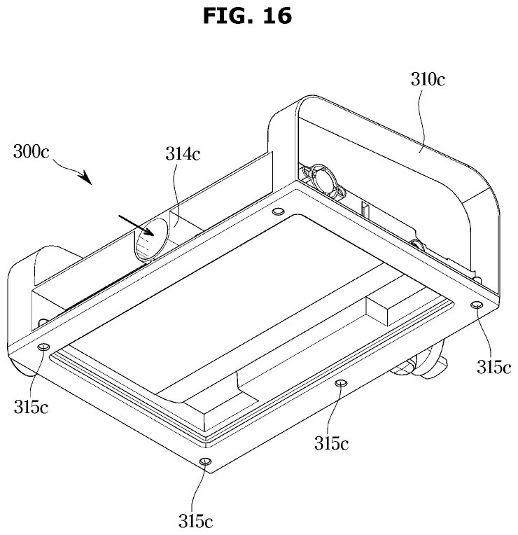

FIG. 15 is a front perspective view illustrating an exhaust portion of a cleaning device according to still another embodiment of the disclosure. FIG. 16 is a bottom perspective view of FIG. 15. FIGS. 17 and 18 are views illustrating an operation of an exhaust portion, which is seen from the front with a bottom frame shown in FIG. 15 removed.

An exhaust portion 300c of a cleaning device according to still another embodiment of the disclosure is detachably coupled to a dust collecting station 3b.

Referring to FIGS. 15 and 16, the exhaust portion 300c may include an accommodation frame 310c, and a communication portion 314c may be formed at a front side of the accommodation frame 310c.

Because the exhaust portion 300c of the cleaning device according to the still another embodiment of the disclosure is detachably coupled to the dust collecting station 3b, the communication portion 314c into which air exhausted from the dust collecting station 3b is introduced is separately formed. The communication portion 314c may be provided in a tubular shape with an interior that is open such that air flows. However, the shape of the communication portion 314c is not limited thereto.

In addition, the exhaust portion 300c may include a bottom frame 390c mounted on the bottom surface of the accommodation frame 310c. The bottom frame 390c may include a plurality of coupling portions 315c. With such a configuration, the separated exhaust portion 300c may be provided to be coupled to a base of the dust collecting station 3b using a separate fastening member. However, the bottom frame 390c is not limited thereto and may be formed integrally with the accommodation frame 310c.

Hereinafter, an operation of the exhaust portion 300c of the cleaning device according to the still another embodiment of the disclosure will be described with reference to FIGS. 17 and 18.

A moving plate 350c of the exhaust portion 300c has opposite ends respectively coupled to the driving shaft 330c and the support shaft 340c to slidably move with respect to the accommodation frame 310c and an intake portion 50c.

A motor portion 320c provides power to the driving shaft 330c to rotate the driving shaft 330b, and as the driving shaft 330b rotates, the moving plate 350c may move leftward and rightward.

The moving plate 350c may include a cutter member and a dust removal member formed on the upper surface thereof to remove dust and foreign substances while sweeping a roller 52c, a cleaning roller 53 of the intake portion 50c.

In addition, although not shown separately, the moving plate 350c may be provided to be elastically pressed against the roller 52c, the cleaning roller 53c of the intake portion 50c such that the intake portion 50c is cleaned with a certain adhesion force.

Hereinafter, a cleaning device 1′ according to still another embodiment of the disclosure will be described. Components other than a base 200 and an exhaust portion 500 to be described below are the same as those of the cleaning device 1 according to the above-described embodiment, and thus the same description will be omitted.

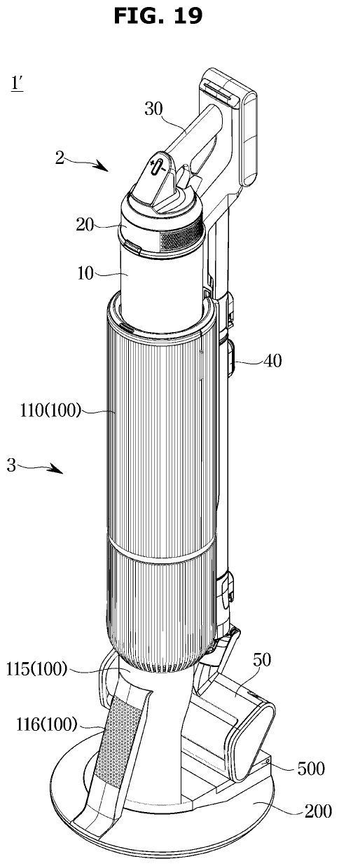

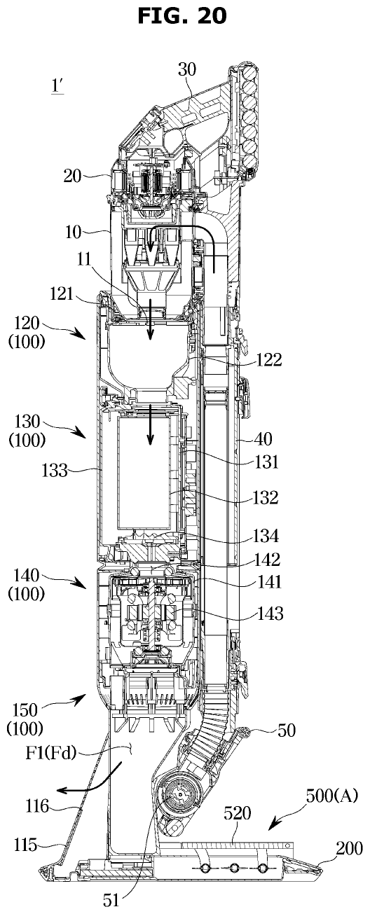

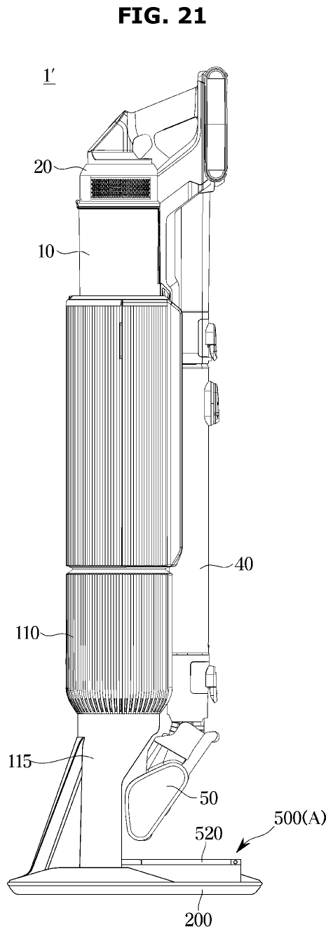

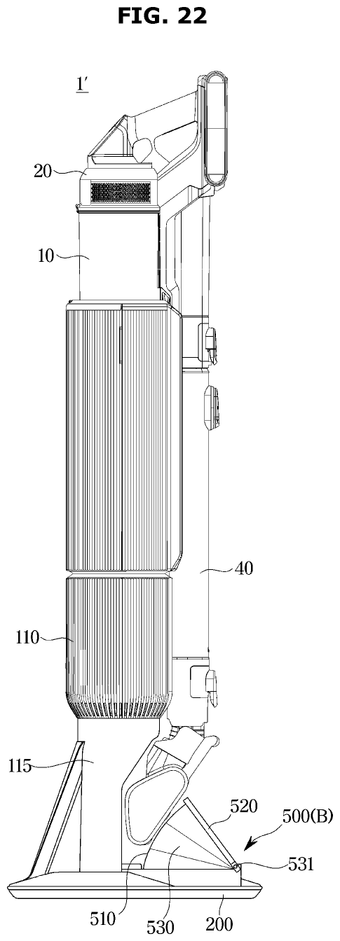

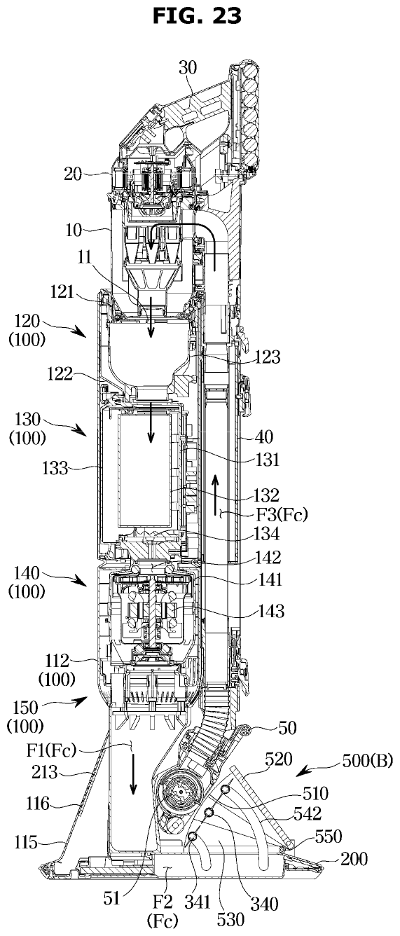

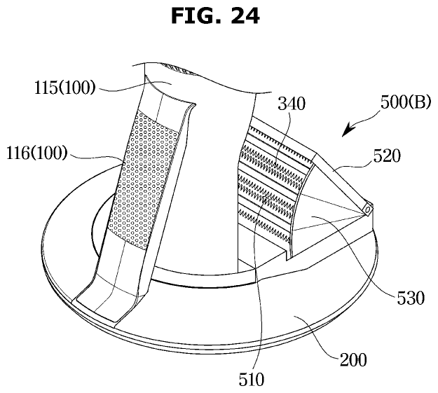

FIG. 19 is a perspective view illustrating a state in which a vacuum cleaner is coupled to a dust collecting station of a cleaning device according to still another embodiment of the disclosure. FIG. 20 is a side cross-sectional view of FIG. 19. FIG. 21 is a view illustrating a state in which the exhaust portion of the dust collecting station shown in FIG. 19 is inserted into a base. FIG. 22 is a view illustrating a state in which the exhaust portion of the dust collecting station shown in FIG. 19 is withdrawn from a base. FIG. 23 is a side cross-sectional view of FIG. 22. FIG. 24 is a view showing an exhaust portion and a base of the dust collecting station of FIG. 19.

Referring to FIGS. 19 to 24, a driving device 143 of a dust collecting station 3 may provide power to generate a suction force. The driving device 143 may include components, such as a motor and a fan. That is, a suction airflow may be formed in the dust collecting station 3 by the driving device 143.

The suction airflow may have a change in a flow depending on a flow path of the cleaning device 1′ that is varied according to the position of the exhaust portion 500 to be described below. When the exhaust portion 500 is arranged in a first position A, the cleaning device 1′ forms a discharge flow path Fd in which the suction airflow is discharged to the outside of the housing 110 (see FIG. 20).

The dust collecting station 3 may include a first flow path F1 formed inside a station body 110 and connected from a seating portion 120 to a suction device 140. In detail, the first flow path F1 may have one end provided with a seating portion opening 121, and the other end connected to a second flow path F2 to be described below.

In addition, when the exhaust portion 500 is arranged in a second position B, the cleaning device 1 forms a circulation flow path Fc in which the suction airflow circulates through the dust collecting station 3 and the vacuum cleaner 2 (see FIG. 23).

A filter portion 150 may include a filter therein. When the driving device 143 of the suction device 140 forms an airflow that suctions air from the upper side of the dust collecting station 3, the air passed through the suction device 140 may pass through the filter portion 150. The filter portion 150 may be provided at a lower side of a collecting portion 130 to filter the air passed through the collecting portion 130. The air passed through the filter portion 150 may flow into the base 200 or may be discharged to the outside of a housing 110 through a discharge port 116.

The dust collecting station 3 may include the discharge port 116 through which the suction airflow is discharged to the outside of the housing 110. The discharge port 116 may be preferably arranged on a connecting portion 115, but the disclosure is not limited thereto, and the discharge port 116 may be arranged on the housing 110.

In addition, the discharge port 116 may not be defined as a specific component, and may be formed by at least a portion of the housing 110 that communicates with the outside.

The discharge port 116 may be arranged below the filter portion 150 such that the suction airflow passes through the filter portion 150 and then flows out of the housing 110 through the discharge port 116. This is to prevent a contaminated airflow from being discharged to the outside of the housing 110.

When the cleaning device 1 forms the discharge flow path Fd, the suction airflow may be caused to flow on the first flow path F1 and discharged to the outside of the housing 110 through the discharge port 116.

The base 200 may be provided at a lower side of the station body 100 to support the station body 100.

The dust collecting station 3 may include the exhaust portion 500 that is arranged to be accommodated in the base 200 and configured to exhaust the suction airflow to the intake portion 50 of the vacuum cleaner 2 to separate foreign substances remaining in the intake portion 50 from the intake portion 50.

The exhaust portion 500 may include an exhaust hole 510 for exhausting air to the intake portion 50. The exhaust portion 500 may be provided to be moved between the first position A in which the exhaust hole 510 is inserted into the base 200 and the second positions B in which the exhaust hole 510 protrudes to the outside of the base 200 such that the suction airflow is exhausted to the intake portion 50 through the exhaust hole 510. Details thereof will be described below in detail.

Referring FIGS. 20 and 21, when the exhaust portion 500 is arranged in the first position A, the exhaust portion 500 may be inserted into the base 200. In detail, the exhaust hole (510 in FIG. 23) may be provided to be inserted into the base 200.

The exhaust portion 500 may be provided to be arranged in the first position A in a state in which the dust collecting station 3 is not operated. The exhaust portion 500 may, in response to the suction device 140 being driven or a signal being input to an input unit (170 in FIG. 9) by a user, move to the second position B, and in response to the driving of the suction device 140 being terminated, move from the second position B to the first position A again.

The input unit 170 may be arranged on the dust collecting station 3 or may be implemented through an application interoperating with the dust collecting station 3.

The exhaust portion 500 may include an exhaust portion cover 520 that forms an upper surface of the exhaust portion 500. The exhaust portion 500 may be provided such that only the exhaust portion cover 520 is exposed to the outside when the exhaust portion 500 is arranged in the first position A. Preferably, the exhaust portion cover 520 may be provided to be arranged on the same line as the upper surface of the base 200 when the exhaust portion 500 is arranged in the first position A.

The exhaust portion cover 520 may be provided to form at least a portion of the external appearance of the base 200 when the exhaust portion 500 is arranged in the first position A.

Components other than the exhaust portion cover 520 of the exhaust portion 500 may be provided to be inserted into the base 200 when the exhaust portion 500 is arranged in the first position A.

When the exhaust portion 500 is arranged in the first position A, the cleaning device 1′ may be provided such that the suction airflow flows through the discharge flow path Fd. As will be described below, when the exhaust portion 500 is arranged in the second position B, the suction airflow may be provided to flow through the second flow path F2 disposed inside the base 200 to a third flow path F3 formed inside the vacuum cleaner 2 and then circulate to the first flow path F1 again. However, when the exhaust portion 500 is arranged in the first position A, the second flow path F2 and the third flow path F3 are provided to be separated from each other, so that the suction airflow may be discharged to the outside of the dust collecting station 3 through the discharge port 116 formed on the first flow path F1.

Referring to FIGS. 22 to 24, when the exhaust portion 500 is arranged in the second position B, the exhaust hole 510 may be provided to be arranged outside the base 200.

The exhaust hole 510 may protrude from the base 200 to face an intake port of the intake portion 50.

The dust collecting station 3 may include the second flow path F2 formed inside the base 200 and connected from the first flow path F1 to the exhaust hole 510 of the exhaust portion 500. As described above, the first flow path F1 and the second flow path F2 may be provided to be formed inside the dust collecting station 3.

The other end of the second flow path F2 is provided as the exhaust hole 510, so that when the exhaust portion 500 is arranged in the first position A, the suction airflow may be discharged to the outside through the discharge port 116 rather than flowing to the outside through the second flow path F2, but when the exhaust portion 500 is arranged in the second position B, the exhaust hole 510 is exposed to the outside, so that the suction airflow flowing through the second flow path F2 may be discharged to the outside through the exhaust hole 510.

Because the exhaust hole 510 is provided to face the intake port of the intake portion 50 as described above, the suction airflow discharged to the outside through the exhaust hole 510 may flow toward the intake port of the intake portion 50.

The suction airflow discharged from the exhaust hole 510 may cause foreign substances remaining in the intake portion 50 to be separated from the intake portion 50 together with the suction airflow. Accordingly, the exhaust portion 500 may prevent the intake portion 50 from being contaminated by foreign substances remaining in the intake portion 50.

The vacuum cleaner 2 may include the third flow path F3 connected from the intake portion 50 to the dust collecting container 10 along the extension pipe 40.

The suction airflow discharged from the exhaust portion 500 may be provided to flow along the third flow path F3 together with the foreign substances remaining in the intake portion 50 through the intake portion 50 and then circulate back to the first flow path F1.

That is, the suction airflow formed by the suction device 140 may flow inside the dust collecting station 3 through the first flow path F1 and the second flow path F2, and then flow from the second flow path F2 to the third flow path F3 to flow inside the vacuum cleaner 2, and then flow again into the first flow path F1 to flow inside the dust collecting station 3.

Accordingly, the cleaning device 1′ with the exhaust portion 5000 arranged in the second position B may form the circulation flow path Fc such that the suction airflow circulates through the first flow path F1, the second flow path F2, and the third flow path F3.

The dust collecting station 3 may include an opening and closing member 117 provided to selectively open and close the discharge port 116. As described above, when the cleaning device 1′ forms the discharge flow path Fd, the opening and closing member 117 may be provided to open the discharge port 116.

Conversely, when the cleaning device 1′ forms the circulation flow path Fc, the opening and closing member 117 may be provided to close the discharge port 116.

The opening and closing member 117 maybe provided to maintain the flow pressure in each flow path, when the cleaning device 1 selectively forms the discharge flow path Fd and the circulation flow path Fc.

However, the disclosure is not limited thereto, and the dust collecting station 3 may not include the opening and closing member 117. In this case, the exhaust hole 510 may be provided to have a cross-sectional area larger than that of the discharge port 116. Accordingly, even without a separate opening and closing member, a large amount of suction airflow may be provided to flow to the exhaust hole 510 rather than to the discharge port 116 according to the pressure difference of the air.

The exhaust portion 500 may include a guide 530 that forms at least a portion of the exhaust hole 510 and guides the suction airflow such that the suction airflow flows into the exhaust hole 510.

The guides 530 may be provided to form opposite side ends of the exhaust hole 510 and may be provided as a pair. The guide 530 may be arranged inside the base 200 when the exhaust portion 500 is arranged in the first position A, and may be provided to protrude outside of the base 200 when the exhaust portion 500 is arranged in the second position B.

Although not shown in the drawing, the guide 530, when the exhaust portion 500 is arranged in the first position A, may be folded in a telescopic manner to be rotated about a hinge shaft 531, and when the exhaust portion 500 is arranged in the second position B, may be unfolded to protrude to the outside of the base 200.

The hinge shaft 531 may also be provided as a rotation shaft for the protrusion of the exhaust portion cover 520 as well as the guide 530. That is, the guide 530 together with the exhaust portion cover 520 may be provided to rotate upward of the base 200 about the hinge shaft 531 to protrude upward from the base 200.

Although not shown in the drawings, the exhaust portion 500 may include a driving unit (not shown) for driving the exhaust portion 500 such that the exhaust portion 500 moves from the first position A to the second position B or vice versa. The driving unit (not shown) may include a driving motor and a power transmission member to allow the guide 530 and the exhaust portion cover 520 to move.

However, the disclosure is not limited thereto, and the guide 530 may be provided to be inserted into the base 200 and protruded out of the base 200 in various forms. For example, the guide 530 may be provided to contract and expand in a bellows form to be inserted into the base 200 and protruded from the base 200, or may be provided to be arranged inside the base 200 and raised upward by an elevation device to be protruded outside of the base 200 without a change in shape.

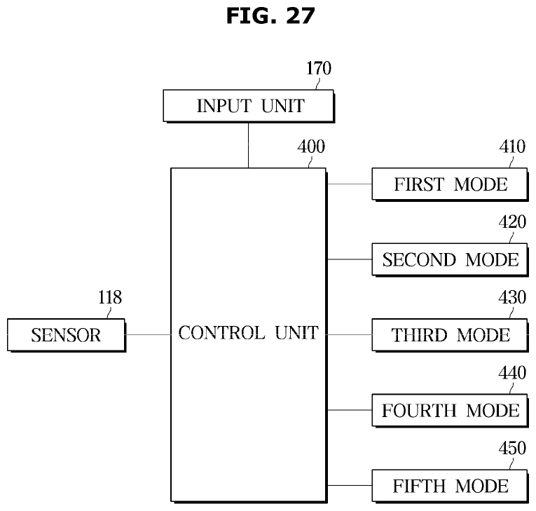

The dust collecting station 3 may include a control unit (400 in FIG. 27) that controls the driving of the suction device 140 and controls the exhaust portion 500 so that the exhaust portion 500 is moved to the first position A or the second position B.

In response to the vacuum cleaner 2 docked onto the dust collecting station 3, the control unit 400 may receive a signal from a sensor (118 in FIG. 24) that determines whether the vacuum cleaner 2 is docked onto the dust collecting station 3 and control the exhaust portion 500 so that the exhaust portion 500 is moved from the first position A to the second position B.

The sensor 118 may be provided as a switch sensor or an optical sensor. The sensor 118 may be arranged approximately on a connecting portion 115 and may be configured to sense the position at which the intake portion 50 is arranged to determine whether the vacuum cleaner 2 is mounted in the correct position on the dust collecting station 3 and transmit a signal to the control unit 400.

However, the disclosure is not limited thereto, and the sensor 118 may be arranged adjacent to the seating portion 120 to sense whether the dust collecting container 10 is seated on the seating portion 120, or may include a plurality of units thereof adjacent to the seating portion 120 and the connecting portion 115 to sense whether the vacuum cleaner 2 is mounted in a correct position on the dust collecting station 3.

In addition, the control unit 400 may, in response to the operation of the suction device 140 being terminated, control the exhaust portion 500 so that the exhaust portion 500 arranged in the second position B is moved to the first position A

However, the disclosure is not limited thereto, and even in response to the vacuum cleaner 2 being docked onto the dust collecting station 3, the control unit 400 may control the exhaust portion 500 so that the exhaust portion 500 remains in the first position A without being moved to the second position B according to an input signal of a user. In addition, in response to a signal for driving the exhaust portion 500 being input by a user, the control unit 400 may control the exhaust portion 500 so that the exhaust portion 500 is moved from the first position A to the second position B.

While the vacuum cleaner 2 is docked onto the dust collecting station 3, in response to an input signal for driving the suction device 140 that is terminated, the control unit 400 may control the exhaust portion 500 so the exhaust portion 500 is moved to the second position B together with the driving of the suction device 140.

That is, the exhaust portion 500 may be provided to be arranged in the first position A when the dust collecting station 3 is driven, and selectively moved from the first position A to the second position B.

Because the exhaust portion 500 is formed to be selectively protruded to the outside of the base 200 as needed, the dust collecting station 3 may achieve slimness, and because the exhaust hole 510 is not exposed to the outside when the suction device 140 is not driven, the exhaust portion 500 may be prevented from being contaminated by the outside.

The exhaust portion 500 may include a cleaning unit 340.

The cleaning unit 340 may be provided to separate dust accumulated in the intake portion 50. In detail, the intake portion 50 includes a cleaning roller 53 for guiding suction of foreign substances, and the cleaning unit 340 may be provided to apply an external force to the cleaning roller 53 such that foreign substances remaining on the cleaning roller 53 are separated from the cleaning roller 53 and flow together with the suction airflow.

The cleaning unit 340 may, in response to the exhaust portion 500 being arranged in the first position A, enter the base 200 and in response to the exhaust portion 500 being arranged in the second position B, protrude out of the base 200.

The cleaning unit 340 may include a cleaner 341 that presses the cleaning roller 53. The cleaner 341 may be preferably arranged on the exhaust hole 510. The cleaning unit 340 may include a rail 542 guiding the movement of the cleaner 341 such that the cleaner 341 is arranged on the exhaust hole 510 when the exhaust portion 500 is arranged in the second position B.

The cleaning unit 340 and the cleaner 341 may be provided in the same configuration as the cleaning unit 340 and the cleaner 341 of the cleaning device 1 according to the embodiment of the disclosure described above, and may be variously configured according to the embodiments described above.

The rail 542 may preferably be formed on the inner side of the guide 530. The cleaner 341 may be provided to slide on the exhaust hole 510 inside the base 200 along the rail 542 formed in the guide 530.

The rail 542 may be provided to be formed in association with the shape of the guide 530 being expanded when the exhaust portion 500 is arranged in the second position B. That is, the rail 542 may be provided in a folded shape together with the guide 530 in response to the exhaust portion 500 being arranged in the first position A and may be provided to form a shape together with the guide 530 in response to the exhaust portion 500 being moved to the second position B.

However, the disclosure is not limited thereto, and the cleaner 341 may be provided to be arranged on the exhaust hole 510 in various ways in response to the exhaust portion 500 being arranged in the second position B.

The exhaust portion 500 may include a sterilization device 550 provided to sterilize the intake portion 50.

The sterilization device 550 may, in response to the exhaust hole 510 being opened toward the intake portion 50, remove contamination caused by foreign substances remaining in the intake portion 50 through the exhaust hole 510.

In detail, the sterilization device 550 may be provided as an ultraviolet (UV) lamp transmitting ultraviolet rays. Ultraviolet rays from the UV lamp may be transmitted through the exhaust hole 510 to the intake portion 50 to remove germs and bacteria propagating in the intake portion 50 due to foreign substances remaining in the intake portion 50.

Ultraviolet rays transmitted from the sterilization device 550 may preferably be in a range of wavelengths of 200 nm to 290 nm.

The UV lamp of the sterilization device 550 may be preferably arranged at a lower side of the hinge shaft 531 to form a wide irradiation angle in the intake portion 50 and transmit ultraviolet rays to the intake portion 50.

The UV lamp may be preferably arranged within a range of about 10 cm from the intake portion, specifically, from the cleaning roller 53 irradiated with ultraviolet rays.

The sterilization device 550 may be provided to be driven by the control unit 400 in response to the exhaust portion 500 being arranged in the second position B.

Hereinafter, the operation of the cleaning unit 340 will be described in detail.

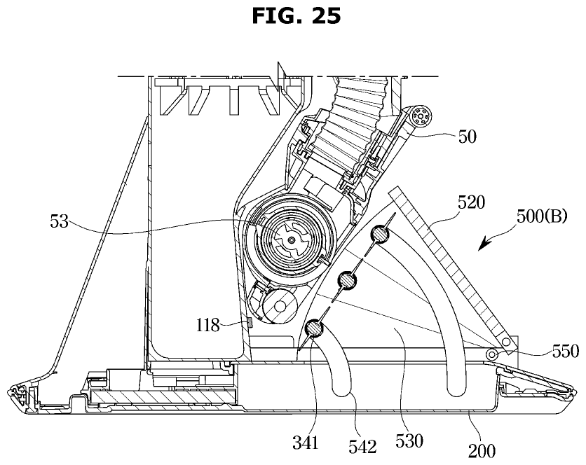

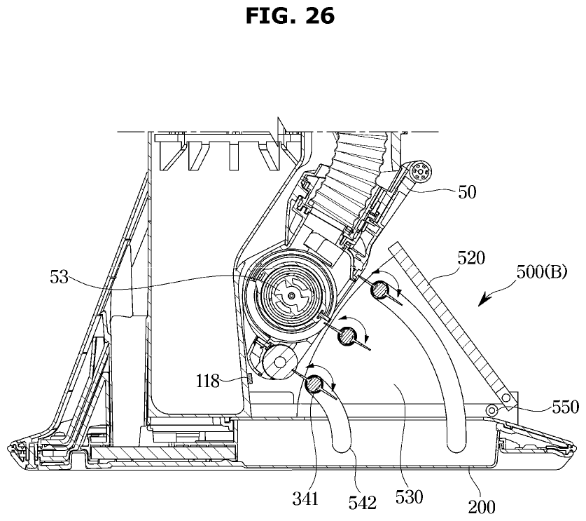

FIG. 25 is a cross-sectional view illustrating an operation of a cleaning unit of the dust collecting station of FIG. 19. FIG. 26 is a cross-sectional view illustrating an operation of a cleaning unit of the dust collecting station of FIG. 19.

Referring to FIGS. 25 and 26, the cleaner 341 of the cleaning unit 340 may be provided in plural. However, the disclosure is not limited thereto, and the number of the cleaners 341 may be variously formed.

The cleaners 341 may be provided to rotate to press the cleaning roller 53. Each of the cleaners 341 may be provided to be rotated in connection with a motor (not shown). Each of the cleaners 341 may be provided to be rotatable by receiving rotational force generated from a motor (not shown) through a gear unit (not shown).

The plurality of cleaners 341 may be provided to be moved at the same time as the exhaust portion 500 is moved from the first position A to the second position B through a frame (not shown). That is, when the frame (not shown) with at least a portion thereof inserted into the rail 542 is moved along the rail 542, and then the plurality of cleaners 341 supported by the frame (not shown) are moved together with the frame (not shown) to cause the exhaust portion 500 to be arranged in the second position B, the plurality of cleaners 341 may be provided to be arranged on the exhaust hole 510.

The cleaner 341 may rotate to come in contact with the cleaning roller 53 so that foreign substances remaining on the cleaning roller 53 may be separated. That is, while the suction airflow is flowing to the intake portion 50 through the exhaust hole 510, the cleaner 341 is rotated to press the cleaning roller 53 so that foreign substances on the cleaning roller 53 are separated and the separated foreign substances are caused to flow along the third flow path F3 together with the suction airflow to be collected in the collecting portion 130.

As described above, the driving of the cleaning unit 340 including the cleaner 341 may be provided in the same manner as one of the cleaning units disclosed in the above-described embodiments of the disclosure.

Hereinafter, a control flow of the cleaning device 1′ according to the another embodiment of the disclosure will be described.

FIG. 27 is a schematic block diagram illustrating a control flow of the dust collecting station shown in FIG. 19.

As described above, the dust collecting station 3 may include the control unit 400 configured to control the driving of the suction device 140 based on information collected from the sensor 118, and control the exhaust portion 500 so that the exhaust portion 500 is moved to the first position A or the second position B.

The control unit 400 may control the dust collecting station 3 so that the dust collecting station 3 is driven in various modes 410, 420, 430, 440, and 450 based on a signal input by the user through the input unit 170.

The control unit 400 may control the dust collecting station 3 so that the dust collecting station 3 is driven in the first mode 410.

In the first mode 410, the control unit 400 may, in response to the vacuum cleaner 2 docked onto the dust collecting station 3, control the exhaust portion 500 so that the exhaust portion 500 is moved from the first position A to the second position B based on a signal received from the sensor 118 that determines whether the vacuum cleaner 2 is docked.

Thereafter, the control unit 400 may drive the suction device 140 and control the dust collecting station 3 so the airflow generated from the suction device 140 circulates between the dust collecting station 3 and the vacuum cleaner 2.

The control unit 400 may drive the suction device 140 and control the cleaning unit 340 so that the cleaning unit 340 is driven for foreign substances remaining in the intake portion 50 to be moved to the collecting portion 130 while circulating between the vacuum cleaner 2 and the dust collecting station 3 together with the suction airflow.

The control unit 400 may, in response to the exhaust portion 500 being arranged in the second position B, drive the sterilization device 550 such that ultraviolet rays are transmitted to the intake portion 50 through the UV lamp.

The control unit 400 may, in response to the sterilizing device 550 being driven, control the vacuum cleaner 2 so that the cleaning roller 53 of the vacuum cleaner 2 rotates a predetermined angle for ultraviolet rays from the sterilizing device 550 are transmitted to the intake portion 50, particularly, to the entire area of the cleaning roller 53.

For example, the control unit 400 may, in response to the vacuum cleaner 2 being docked in the dust collecting station 3, control the vacuum cleaner 2 so that the sterilization device 550 is driven at a position corresponding to the position of the cleaning roller 53 and after a predetermined time elapses, the cleaning roller 53 is rotated 180 degrees.

The control unit 400 may preferably control the sterilization device 550 and the vacuum cleaner 2 such that the cleaning roller 53 is irradiated with ultraviolet rays for about 30 seconds in each irradiation area by the sterilization device 550.

That is, the control unit 400 may control the vacuum cleaner 2 so that the cleaning roller 53 is rotated approximately 180 degrees 30 seconds after the driving of the sterilization device 550, and in response to the sterilization device 550 being driven for 30 minutes after the rotation of 180 degrees of the cleaning roller 53, control the dust collecting station 3 so that the driving of the sterilization device 550 is stopped.

In addition, in order to remove foreign substances remaining on the entire area of the cleaning roller 53 even during the drive of the cleaning unit 340, the control unit 400 may control the vacuum cleaner 2 so that the cleaning roller 53 of the vacuum cleaner 2 is rotated a predetermined angle in the driving of the cleaning unit 340.

The order in which the cleaning unit 340 and the sterilization device 550 are driven by the control unit 400 may be arbitrary, and the dust collecting station 3 may be controlled so that the cleaning unit 340 and the sterilization device 550 are simultaneously driven.

The control unit 400 may control the dust collecting station 3 so that the operation of the suction device 140 is terminated after a predetermined time elapses. In addition, the disclosure is not limited thereto, and the control unit 400 may control the dust collecting station 3 so that the operation of the suction device 140 is terminated, in response to a signal input into the input unit 170 by a user to terminate the operation of the suction device 140 during the drive of the suction device 140,

The control unit 400 may, in response to the driving of the suction device 140 being terminated, control the exhaust portion 500 so that the exhaust portion 500 arranged in the second position B is moved to the first position A.

Preferably, the control unit 400 may, in response to the user not selecting any driving mode and not inputting a signal into the input unit 170, control the dust collecting station 3 so that the dust collecting station 3 is basically driven in the first mode 410.

In the second mode 420, the control unit 400 may, in response to the vacuum cleaner 2 being docked onto the dust collecting station 3, control the dust collecting station 3 so that the exhaust portion 500 remains in the first position A without being moved to the second position B even with the vacuum cleaner 2 being docked onto the dust collecting station 3, based on a signal received from the sensor 118 for determining whether the vacuum cleaner 2 is docked.

In this case, the control unit 400 may control the dust collecting station 3 so that only the suction device 140 is driven without driving the exhaust portion 500.

Thereafter, the control unit 400 may control the dust collecting station 3 so that the operation of the suction device 140 is terminated after a predetermined time elapses. In addition, the disclosure is not limited thereto, and the control unit 400 may control the dust collecting station 3 so that the operation of the suction device 140 is terminated in response to the user inputting a signal to terminate the operation of the suction device 140 into the input unit 170 during the drive of the suction device 140.

In the third mode 430, the control unit 400 may, in response to the vacuum cleaner 2 being docked onto the dust collecting station 3, control the exhaust portion 500 so that exhaust portion 500 is moved from the first position A to the second position B based on a signal received from the sensor 118 for determining whether the vacuum cleaner 2 is docked.

Thereafter, the control unit 400 may drive the suction device 140 and control the dust collecting station 3 so that the airflow generated from the suction device 140 circulates between the dust collecting station 3 and the vacuum cleaner 2.

The control unit 400 may control the dust collecting station 3 so that only the suction device 140 is driven without driving the cleaning unit 340 and the sterilization device 550 during the driving of the suction device 140 for the suction airflow generated from the suction device 140 to be circulated between the vacuum cleaner 2 and the dust collecting station 3.

The control unit 400 may control the dust collecting station 3 so that the operation of the suction device 140 is terminated after a predetermined time elapses. In addition, the disclosure is not limited thereto, and the control unit 400 may control the dust collecting station 3 so that the operation of the suction device 140 is terminated in response to a signal input into the input unit 170 by the user to terminate the operation of the suction device 140 during the driving of the suction device 140.

The control unit 400 may, in response to the driving of the suction device 140 being terminated, control the exhaust portion 500 so that the exhaust portion 500 arranged in the second position B is moved to the first position A.

In the fourth mode 440, the control unit 400 may, in response to the vacuum cleaner 2 being docked onto the dust collecting station 3, control the exhaust portion 500 so that the exhaust portion 500 is moved from the first position A to the second position B based on a signal received from the sensor 118 for determining whether the vacuum cleaner 2 is docked.

Thereafter, the control unit 400 may drive the suction device 140 and control the dust collecting station 3 so that the airflow generated from the suction device 140 circulates between the dust collecting station 3 and the vacuum cleaner 2.

The control unit 400 may control the dust collecting station 3 so that only the cleaning unit 340 is driven during the drive of the suction device 140. The control unit 400 may control the dust collecting station 3 so that the cleaning unit 340 is driven for foreign substances remaining in the intake portion 50 to be separated. In this case, in order for the cleaning unit 340 to separate foreign substances remaining on the entire area of the cleaning roller 53, the control unit 400 may control the vacuum cleaner 2 so that the cleaning roller 53 of the vacuum cleaner 2 is rotated at a predetermined angle during the driving of the cleaning unit 340.

The control unit 400 may control the dust collecting station 3 so that the operation of the suction device 140 is terminated after a predetermined time elapses. In addition, the disclosure is not limited thereto, and the control unit 400 may control the dust collecting station 3 so that the operation of the suction device 140 is terminated in response to a signal input into the input unit 170 by the user to terminate the operation of the suction device 140 during the driving of the suction device 140.

The control unit 400 may, in response to the driving of the suction device 140 being terminated, control the exhaust portion 500 so that the exhaust portion 500 arranged in the second position B is moved to the first position A.

In the fifth mode 450, the control unit 400 may, in response to the vacuum cleaner 2 being docked onto the dust collecting station 3, control the exhaust portion 500 so that the exhaust portion 500 is moved from the first position A to the second position B based on a signal received from the sensor 118 for determining whether the vacuum cleaner 2 is docked.

Thereafter, the control unit 400 may drive the suction device 140 and control the dust collecting station 3 so that the airflow generated from the suction device 140 circulates between the dust collecting station 3 and the vacuum cleaner 2.

The control unit 400 may control the dust collecting station 3 so that only the sterilization device 550 is driven during the driving of the suction device 140. The control unit 400 may control the dust collecting station 3 so that the sterilization device 550 is driven to prevent the intake portion 50 from being contaminated by foreign substances remaining in the intake portion 50. In this case, in order for the entire area of the cleaning roller 53 to be irradiated with ultraviolet rays by the sterilization device 350, the control unit 400 may control the vacuum cleaner 2 so that the cleaning roller 53 of the vacuum cleaner 2 is rotated a predetermined angle during the driving of the sterilization device 550.

The control unit 400 may control the dust collecting station 3 so that the operation of the suction device 140 is terminated after a predetermined time elapses. In addition, the disclosure is not limited thereto, and the control unit 400 may control the dust collecting station 3 so that the operation of the suction device 140 is terminated in response to a signal input into the input unit 170 by the user to terminate the operation of the suction device 140 during the driving of the suction device 140.

The control unit 400 may, in response to the driving of the suction device 140 being terminated, control the exhaust portion 500 so that the exhaust portion 500 arranged in the second position B is moved to the first position A.

The control unit 400 may drive the dust collecting station 3 in various modes other than the above-described modes 410, 420, 430, 440, and 450. In addition, the disclosure is not limited thereto, and the control unit 400 may control the dust collecting station 2 so that the dust collecting station 3 is driven in an additional mode that combines the respective modes 410, 420, 430, 440, and 450.

Hereinafter, a cleaning device 1′ according to another embodiment of the disclosure will be described. Components other than a sterilizing device 560 of the cleaning device 1′ to be described below are the same as those of the cleaning device 1 according to the above-described embodiments, and thus the same descriptions will be omitted.

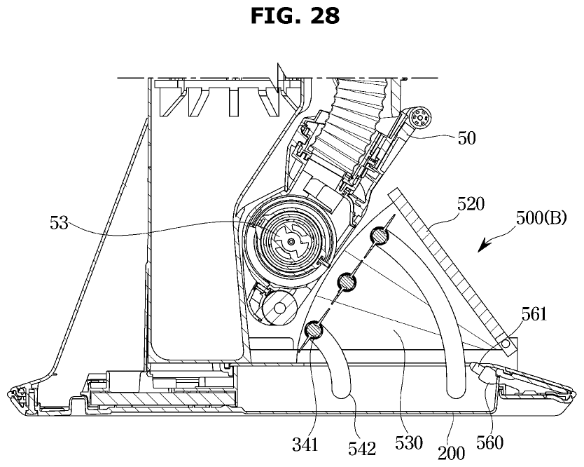

FIG. 28 is a cross-sectional view illustrating a base and an exhaust portion of the cleaning device according to the still another embodiment of the disclosure.

The exhaust portion 500 may include a sterilization device 560 provided to sterilize the intake portion 50.

In response to the exhaust hole 510 being open toward the intake portion 50, the sterilization device 560 may be provided to remove contamination caused by foreign substances remaining in the intake portion 50 through the exhaust hole 510.

In detail, the sterilization device 560 may include a spray nozzle 561 for spraying high-temperature steam. The spray nozzle 561 may be provided to spray the intake portion 50 with high-temperature steam generated by a tank (not shown) in which water is stored and a heater (not shown) heating the water stored in the tank (not shown) of the sterilization device 560. The high-temperature steam from the spray nozzle 561 may be sprayed to the intake portion 50 through the exhaust hole 510 to remove germs and bacteria propagating in the intake portion 50 due to foreign substances remaining in the intake portion 50.

The steam sprayed by the spray nozzle 561 may remain in the intake portion 50 as moisture, but because the suction airflow is provided to flow through the exhaust hole 510, the moisture remaining in the intake portion 50 may be completely removed by the suction airflow.

As is apparent from the above, the cleaning device including the vacuum cleaner and the dust collecting station can perform cleaning on the intake portion without a separate action by mounting the vacuum cleaner on the dust collecting station.

The cleaning device including the vacuum cleaner and the dust collecting station can keep the intake portion always clean by simultaneously emptying the dust collecting container of the vacuum cleaner and cleaning the intake portion.

The cleaning device including the vacuum cleaner and the dust collecting station can improve the cleaning performance of the intake portion by cleaning the intake portion with clean air that passed through the filter portion of the dust collecting station.

The cleaning device including the vacuum cleaner and the dust collecting station can maintain the dust collecting performance of the dust collecting container for a long time by directly collecting dust separated from the intake portion into the collecting portion of the dust collecting station rather than into the dust collecting container of the vacuum cleaner.

Although few embodiments of the disclosure have been shown and described, the above embodiment is illustrative purpose only, and it would be appreciated by those skilled in the art that changes and modifications may be made in these embodiments without departing from the principles and scope of the disclosure, the scope of which is defined in the claims and their equivalents.

What is claimed is:

1. A cleaning device comprising:
a vacuum cleaner including
an intake portion to suction foreign substances into the vacuum cleaner, and
a dust collecting container to collect the foreign substances through centrifugal rotation separation; and
a dust collecting station connectable to the dust collecting container and configured to remove the foreign substances collected in the dust collecting container, the dust collecting station including:
a station body,
a seating portion including an opening to communicate with an inside of the dust collecting container while the dust collecting station is connected to the dust collecting container,
a suction device configured to generate a suction airflow so that the foreign substances in the dust collecting container are suctioned through the opening of the seating portion,
a collecting portion arranged between the seating portion and the suction device and configured to collect the foreign substances that are suctioned through the opening of the seating portion,
a base provided at a lower side of the station body to support the station body and configured to allow the suction airflow to flow therein, and
an exhaust portion connected to the base and formed to allow the suction airflow therein, wherein the exhaust portion includes an exhaust hole through which the suction airflow is exhausted to the intake portion so that the foreign substances remaining on the intake portion are separated from the intake portion.

2. The cleaning device of claim 1, wherein a circulation flow path extends along the exhaust portion, the station body, the base, and the vacuum cleaner.

3. The cleaning device of claim 1, wherein the exhaust portion includes:
an accommodation frame on which the intake portion is seated;
a motor portion mounted on the accommodation frame; and
a cleaning unit having one end coupled to the motor portion to separate dust accumulated on the intake portion, the cleaning unit arranged inside the accommodation frame to face a bottom surface of the intake portion.

4. The cleaning device of claim 3, wherein the cleaning unit includes:
a rotation shaft configured to rotate by receiving power from the motor portion; and
a brush extending in a radial direction with respect to the rotation shaft and provided to rotate, the brush provided to separate dust on the bottom surface of the intake portion.

5. The cleaning device of claim 4, wherein the cleaning unit includes a cleaner including the rotation shaft and the brush, and
the cleaner being provided in plural to sweep off the dust in the intake portion.

6. The cleaning device of claim 3, wherein the cleaning unit includes:
a drive shaft configured to rotate by receiving power from the motor portion;
a support shaft arranged in parallel with the driving shaft and fixed to the accommodation frame; and
a moving plate allowing the driving shaft and the support shaft to pass therethrough and provided to be in contact with the intake portion, the moving plate provided to be slidably movable with respect to the accommodation frame according to rotation of the driving shaft.

7. The cleaning device of claim 6, wherein the moving plate includes a cutting member mounted on one surface of the moving plate that faces the intake portion.

8. The cleaning device of claim 2, wherein the base includes:
a communication frame including a communication flow path forming a part of the circulation flow path; and
a support frame connected to the communication frame to be in contact with an installation surface of the dust collecting station.

9. The cleaning device of claim 8, wherein the dust collecting station further includes a filter provided at a lower side of the collecting portion to filter air that has passed through the collecting portion, and
the air that has passed through the filter is introduced into the communication flow path of the base.

10. The cleaning device of claim 9, wherein the exhaust portion includes an accommodation frame in which the intake portion is accommodated, the accommodation frame integrally formed with the communication frame for air to circulate.

11. The cleaning device of claim 10, wherein the accommodation frame includes a front seating portion that is bent to allow a portion of a front side of the intake portion to be seated thereon.

12. The cleaning device of claim 1, wherein the exhaust portion is provided to be detachable with respect to the base.

13. The cleaning device of claim 1, wherein the exhaust portion is provided to move between a first position in which the exhaust hole is inserted into the base and a second position in which the exhaust hole protrudes outside the base such that the suction airflow is exhausted to the intake portion.

14. The cleaning device of claim 13, wherein the dust collecting station includes:
a first flow path formed inside the station body and connected from the seating portion to the suction device; and
a second flow path connected from the suction device to the base,
wherein one end of the second flow path is formed in the exhaust hole.

15. The cleaning device of claim 13, wherein the exhaust portion further includes a cover forming an upper surface of the exhaust portion, and
the cover forms at least a portion of the base when the exhaust portion is arranged in the first position.
